SEQUENCE LISTING (21 sequences)

SEQ ID NO: 1 - DNA, 834 bp, Homo Sapiens
atgaacgcccctctcggtggaatctggctctggctccctctgctcttgacctggctcacccccgaggtcaactcttcatggtggtacatgagagctacaggtggctcctccagggtgatgtgcgataatgtgccaggcctggtgagcagccagcggcagctgtgtcaccgacatccagatgtgatgcgtgccattagccagggcgtggccgagtggacagcagaatgccagcaccagttccgccagcaccgctggaattgcaacaccctggacagggatcacagcctttttggcagggtcctactccgaagtagtcgggaatctgcctttgtttatgccatctcctcagctggagttgtatttgccatcaccagggcctgtagccaaggagaagtaaaatcctgttcctgtgatccaaagaagatgggaagcgccaaggacagcaaaggcatttttgattggggtggctgcagtgataacattgactatgggatcaaatttgcccgcgcatttgtggatgcaaaggaaaggaaaggaaaggatgccagagccctgatgaatcttcacaacaacagagctggcaggaaggctgtaaagcggttcttgaaacaagagtgcaagtgccacggggtgagcggctcatgtactctcaggacatgctggctggccatggccgacttcaggaaaacgggcgattatctctggaggaagtacaatggggccatccaggtggtcatgaaccaggatggcacaggtttcactgtggctaacgagaggtttaagaagccaacgaaaaatgacctcgtgtattttgagaattctccagactactgt

SEQ ID NO: 2 - protein, 278 aa, Homo Sapiens
MNAPLGGIWLWLPLLLTWLTPEVNSSWWYMRATGGSSRVMCDNVPGLVSSQRQLCHRHPDVMRAISQGVAEWTAECQHQFRQHRWNCNTLDRDHSLFGRVLLRSSRESAFVYAISSAGVVFAITRACSQGEVKSCSCDPKKMGSAKDSKGIFDWGGCSDNIDYGIKFARAFVDAKERKGKDARALMNLHNNRAGRKAVKRFLKQECKCHGVSGSCTLRTCWLAMADFRKTGDYLWRKYNGAIQVVMNQDGTGFTVANERFKKPTKNDLVYFENSPDYC

SEQ ID NO: 3 - protein, 360 aa, Homo Sapiens
MNAPLGGIWLWLPLLLTWLTPEVNSSWWYMRATGGSSRVMCDNVPGLVSSQRQLCHRHPDVMRAISQGVAEWTAECQHQFRQHRWNCNTLDRDHSLFGRVLLRSSRESAFVYAISSAGVVFAITRACSQGEVKSCSCDPKKMGSAKDSKGIFDWGGCSDNIDYGIKFARAFVDAKERKGKDARALMNLHNNRAGRKAVKRFLKQECKCHGVSGSCTLRTCWLAMADFRKTGDYLWRKYNGAIQVVMNQDGTGFTVANERFKKPTKNDLVYFENSPDYCIRDREAGSLGTAGRVCNLTSRGMDSCEVMCCGRGYDTSHVTRMTKCGCKFHWCCAVRCQDCLEALDVHTCKAPKNADWTTAT

SEQ ID NO: 4 - protein, 360 aa, Pan troglodytes
MNAPLGGIWLWLPLLLTWLTPEVNSSWWYMRATGGSSRVMCDNVPGLVSSQRQLCHRHPDVMRAISQGVAEWTAECQHQFRQHRWNCNTLDRDHSLFGRVLLRSSRESAFVYAISSAGVVFAITRACSQGEVKSCSCDPKKMGSAKDSKGIFDWGGCSDNIDYGIKFARAFVDAKERKGKDARALMNLHNNRAGRKAVKRFLKQECKCHGVSGSCTLRTCWLAMADFRKTGDYLWRKYNGAIQVVMNQDGTGFTVANERFKKPTKNDLVYFENSPDYCIRDREAGSLGTAGRVCNLTSRGMDSCEVMCCGRGYDTSHVTRMTKCGCKFHWCCAVRCQDCLEALDVHTCKAPKNADWTTPT

SEQ ID NO: 5 - protein, 360 aa, Macaca sp.; identical to SEQ ID NO: 4

SEQ ID NO: 6 - protein, 360 aa, Gorilla sp.; identical to SEQ ID NO: 4

SEQ ID NO: 7 - protein, 360 aa, Papio sp.; identical to SEQ ID NO: 4

SEQ ID NO: 8 - protein, 360 aa, Mus musculus
MNVPLGGIWLWLPLLLTWLTPEVSSSWWYMRATGGSSRVMCDNVPGLVSRQRQLCHRHPDVMRAIGLGVAEWTAECQHQFRQHRWNCNTLDRDHSLFGRVLLRSSRESAFVYAISSAGVVFAITRACSQGELKSCSCDPKKKGSAKDSKGTFDWGGCSDNIDYGIKFARAFVDAKERKGKDARALMNLHNNRAGRKAVKRFLKQECKCHGVSGSCTLRTCWLAMADFRKTGDYLWRKYNGAIQVVMNQGGTGFTVANKRFKKPTKNDLVYFENSPDYCIRDREAGSLGTAGRVCNLTSRGMDSCEVMCCGRGYDTSHVTRMTKCECKFHWCCAVRCQDCLEALDVHTCKAPKSADWATPT

SEQ ID NO: 9 - protein, 360 aa, Rattus sp.
MNVPLGGIWLWLPLLLTWLTPEVSSSWWYMRATGGSSRVMCDNVPGLVSRQRQLCHRHPDVMRAIGLGVAEWTAECQHQFRQHRWNCNTLDRDHSLFGRVLLRSSRESAFVYAISSAGVVFAITRACSQGELKSCSCDPKKKGSGKDSKGTFDWGGCSDNIDYGIKFARAFVDAKERKGKDARALMNLHNNRAGRKAVKRFLKQECKCHGVSGSCTLRTCWLAMADFRKTGDYLWRKYNGAIQVVMNQDGTGFTVANKRFKKPTKNDLVYFENSPDYCIRDREAGSLGTAGRVCNLTSRGMDSCEVMCCGRGYDTSHVTRMTKCECKFHWCCAVRCQDCLEALDVHTCKAPKSADWTTPT

SEQ ID NO: 10 - protein, 350 aa, Danio rerio [sequence omitted]

SEQ ID NO: 11 - DNA, 3186 bp, Homo sapiens
acagcatggagtggggttacctgttggaagtgacctcgctgctggccgccttggcgctgctgcagcgctctagcggcgctgcggccgcctcggccaaggagctggcatgccaagagatcaccgtgccgctgtgtaagggcatcggctacaactacacctacatgcccaatcagttcaaccacgacacgcaagacgaggcgggcctggaggtgcaccagttctggccgctggtggagatccagtgctcgcccgatctcaagttcttcctgtgcagcatgtacacgcccatctgcctagaggactacaagaagccgctgccgccctgccgctcggtgtgcgagcgcgccaaggccggctgcgcgccgctcatgcgccagtacggcttcgcctggcccgaccgcatgcgctgcgaccggctgcccgagcaaggcaaccctgacacgctgtgcatggactacaaccgcaccgacctaaccaccgccgcgcccagcccgccgcgccgcctgccgccgccgccgcccggcgagcagccgccttcgggcagcggccacggccgcccgccgggggccaggcccccgcaccgcggcggcggcaggggcggtggcggcggggacgcggcggcgcccccagctcgcggcggcggcggtggcgggaaggcgcggccccctggcggcggcgcggctccctgcgagcccgggtgccagtgccgcgcgcctatggtgagcgtgtccagcgagcgccacccgctctacaaccgcgtcaagacaggccagatcgctaactgcgcgctgccctgccacaacccctttttcagccaggacgagcgcgccttcaccgtcttctggatcggcctgtggtcggtgctctgcttcgtgtccaccttcgccaccgtctccaccttccttatcgacatggagcgcttcaagtacccggagcggcccattatcttcctctcggcctgctacctcttcgtgtcggtgggctacctagtgcgcctggtggcgggccacgagaaggtggcgtgcagcggtggcgcgccgggcgcggggggcgctgggggcgcgggcggcgcggcggcgggcgcgggcgcggcgggcgcgggcgcgggcggcccgggcgggcgcggcgagtacgaggagctgggcgcggtggagcagcacgtgcgctacgagaccaccggccccgcgctgtgcaccgtggtcttcttgctggtctacttcttcggcatggccagctccatctggtgggtgatcttgtcgctcacatggttcctggcggccggtatgaagtggggcaacgaagccatcgccggctactcgcagtacttccacctggccgcgtggcttgtgcccagcgtcaagtccatcgcggtgctggcgctcagctcggtggacggcgacccggtggcgggcatctgctacgtgggcaaccagagcctggacaacctgcgcggcttcgtgctggcgccgctggtcatctacctcttcatcggcaccatgttcctgctggccggcttcgtgtccctcttccgcatccgctcggtcatcaagcaacaggacggccccaccaagacgcacaagctggagaagctgatgatccgcctgggcctgttcaccgtgctctacaccgtgcccgccgcggtggtggtcgcctgcctcttctacgagcagcacaaccgcccgcgctgggaggccacgcacaactgcccgtgcctgcgggacctgcagcccgaccaggcacgcaggcccgactacgccgtcttcatgctcaagtacttcatgtgcctagtggtgggcatcacctcgggcgtgtgggtctggtccggcaagacgctggagtcctggcgctccctgtgcacccgctgctgctgggccagcaagggcgccgcggtgggcgggggcgcgggcgccacggccgcggggggtggcggcgggccggggggcggcggcggcgggggacccggcggcggcggggggccgggcggcggcgggggctccctctacagcgacgtcagcactggcctgacgtggcggtcgggcacggcgagctccgtgtcttatccaaagcagatgccattgtcccaggtctgagcggaggggagggggcgcccaggaggggtggggaggggggcgaggagagccaagtgcagcgaagggacacttgatgggctgaggttcccaccccttcacagtgttgattgctattagcatgataatgaactcttaatggtatccattagctgggacttaaatgactcacttagaacaaagtacctggcattgaagcctcccagacccagccccttttcctccattgatgtgcggggagctcctcccgccacgcgttaatttctgttggctgaggagggtggactctgcggcgtttccagaacccgagatttggagccctccctggctgcacttggctgggtttgcagtcagatacacagatttcacctgggagaacctctttttctccctcgactcttcctacgtaaactcccacccctgacttaccctggaggaggggtgaccgccacctgatgggattgcacggtttgggtattcttaatgaccaggcaaatgccttaagtaaacaaacaagaaatgtcttaattatacaccccacgtaaatacgggtttcttacattagaggatgtatttatataattatttgttaaattgtaaaaaaaaaagtgtaaaatatgtatatatccaaagatatagtgtgtacatttttttgtaaaaagtttagaggcttacccctgtaagaacagatataagtattctattttgtcaataaaatgacttttgataaatgatttaaccattgccctctcccccgcctcttctgagctgtcacctttaaagtgcttgctaaggacgcatggggaaaatggacattttctggcttgtcattctgtacactgaccttaggcatggagaaaattacttgttaaactctagttcttaagttgttagccaagtaaatatcattgttgaactgaaatcaaaattgagtttttgcaccttccccaaagacggtgtttttcatgggagctcttttctgatccatggataacaactctcactttagtggatgtaaatggaacttctgcaaggcagtaattccccttaggccttgttatttatcctgcatggtatcactaaaggtttcaaaaccctg

SEQ ID NO: 12 - protein, 694 aa, Homo sapiens
MEWGYLLEVTSLLAALALLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTAAPSPPRRLPPPPPGEQPPSGSGHGRPPGARPPHRGGGRGGGGGDAAAPPARGGGGGGKARPPGGGAAPCEPGCQCRAPMVSVSSERHPLYNRVKTGQIANCALPCHNPFFSQDERAFTVFWIGLWSVLCFVSTFATVSTFLIDMERFKYPERPIIFLSACYLFVSVGYLVRLVAGHEKVACSGGAPGAGGAGGAGGAAAGAGAAGAGAGGPGGRGEYEELGAVEQHVRYETTGPALCTVVFLLVYFFGMASSIWWVILSLTWFLAAGMKWGNEAIAGYSQYFHLAAWLVPSVKSIAVLALSSVDGDPVAGICYVGNQSLDNLRGFVLAPLVIYLFIGTMFLLAGFVSLFRIRSVIKQQDGPTKTHKLEKLMIRLGLFTVLYTVPAAVVVACLFYEQHNRPRWEATHNCPCLRDLQPDQARRPDYAVFMLKYFMCLVVGITSGVWVWSGKTLESWRSLCTRCCWASKGAAVGGGAGATAAGGGGGPGGGGGGGPGGGGGPGGGGGSLYSDVSTGLTWRSGTASSVSYPKQMPLSQV

SEQ ID NO: 13 - protein, 15 aa, Artificial Sequence
  Synthetic peptide
REXAFXXAIXSAXXX

SEQ ID NO: 14 - protein, 14 aa, Artificial Sequence
  Synthetic peptide
RXCXXGXXXXCXCD

SEQ ID NO: 15 - protein, 14 aa, Artificial Sequence
  Synthetic peptide
XXXWXGCXDXXXXX

SEQ ID NO: 16 - protein, 15 aa, Artificial Sequence
  Synthetic peptide
KCHGXSGXCXXXTCW

SEQ ID NO: 17 - protein, 12 aa, Artificial Sequence
  Synthetic peptide
DLVXXXXSPXXC

SEQ ID NO: 18 - DNA, 25 bp, Artificial Sequence
  Synthetic primer
ggatgccagagccctgatgaatctt

SEQ ID NO: 19 - DNA, 24 bp, Artificial Sequence
  Synthetic primer
gccagccagcatgtcctgagagta

SEQ ID NO: 20 - DNA, 21 bp, Artificial Sequence
  Synthetic primer
ggactacaaccgcaccgacct

SEQ ID NO: 21 - DNA, 22 bp, Artificial Sequence
  Synthetic primer
accacaggccgatccagaagac

(19) United States
(12) Patent Application Publication
(10) Pub. No.: US 2011/0319337 A1
(43) Pub. Date: Dec. 29, 2011

(54) Dominant Negative WNT2 Compositions and Methods of Use

(76) Inventors: Dawn Bravo, San Francisco, CA (US); Liang You, San Francisco, CA (US); Zhidong Xu, San Francisco, CA (US); Biao He, South San Francisco, CA (US); David M. Jablons, San Francisco, CA (US)

(21) Appl. No.: 13/142,055

(22) PCT Filed: Dec. 30, 2009
(86) PCT No.: PCT/US09/69863

§ 371 Date: Sep. 8, 2011

Related U.S. Application Data
(60) Provisional application No. 61/141,961, filed on Dec. 31, 2008.

(51) Int. Cl. A61K 38/17; C12N 15/12; C12N 15/63; C12N 1/21; C07K 19/00; C12N 5/10; C12N 5/09; G01N 33/566; C12Q 1/68; A61P 35/00; C07K 14/47; C12N 1/19

(52) U.S. Cl. 514/19.3; 530/395; 536/23.5; 435/320.1; 435/252.3; 435/254.2; 435/325; 435/366; 435/348; 435/252.33; 435/358; 435/369; 435/367; 435/350; 435/370; 435/352; 435/364; 435/375; 435/7.21; 435/6.12; 435/7.23; 435/6.14

(57) ABSTRACT

The present disclosure provides compositions, pharmaceutical preparations, kits and methods for inhibiting cell proliferation by contacting a cell expressing Fzd8 with a truncated Wnt2 polypeptide which acts as a dominant negative inhibitor of Fzd8 signaling. The present disclosure provides compositions, kits and methods for the detection of cancer by determining the level of Fzd8 and/or Wnt2 expression in a cell.

### CROSS REFERENCE TO RELATED APPLICATION

This application claims the benefit of U.S. Provisional Patent Application No. 61/141,961 filed Dec. 31, 2008, which application is incorporated herein by reference in its entirety.

### GOVERNMENT RIGHTS

This invention was made with government support under Federal Grant No. F32CA119636 awarded by the National Cancer Institute. The United States Government has certain rights in this invention.

### BACKGROUND

Lung cancer is the most commonly diagnosed malignancy worldwide and is responsible for over 1 million deaths each year (Smith et al., Semin. Oncol. (2004) 31 (2 Supp 4): 11-15; Parkin et al., CA Cancer J. Clin. (2005) 55(2): 74-108). The high mortality rate of lung cancer is largely due to difficulty of detection. Consequently, lung cancer is often diagnosed in advanced stages for which prognosis remains poor. Lung cancer can be divided into two main histological groups; non-small cell lung cancer (NSCLC) and small-cell lung cancer (SCLC). NSCLC, comprised primarily of adenocarcinomas, squamous cell carcinomas and large-cell carcinomas, is more common and accounts for approximately 80%-85% of all lung cancers. The more aggressive SCLC is less frequent and accounts for the remaining 15-20% (Sekido et al., Biochem. Biophys. Acta (1998) 178(1): 21-59). Current treatment strategies for lung cancer include surgical resection, chemotherapy, radiation therapy, targeted therapy, or a combination of treatments, depending on the type and stage of the disease. Despite advances made in these treatments, lung cancer remains highly lethal, with a 5-year survival rate of less than 15% (Smith et al., supra); hence, new therapeutics are needed.

One of the earliest findings indicating a role of the Wnt pathway in cancer was the discovery that ectopic activation of the Int-1 (Wnt-1) gene caused mammary gland tumor formation in mice (Nusse et al., Cell (1982) 31(1): 99-109). Since then numerous accounts of aberrant activation of Wnt pathway constituents have been implicated in human malignancies, particularly the tumor suppressor genes adenomatosis polyposis coli (APC) and β-catenin. Approximately 85% of all sporadic and hereditary colorectal tumors show loss of APC function resulting in stabilization of β-catenin (Rubinfeld et al., Science (1993) 262(5140): 1731-34; Su et al., Science (1993) 262(5140): 1734-37). The link between stabilized β-catenin and tumorigenesis was strengthened by discoveries of mutations in other components of the destruction complex, causing increased cellular levels of free β-catenin (Korinek et al., Science (1997) 275(5307): 1784-87; Morin et al., Science (1997) 275(5307):1787-90; Rubinfeld et al., Science (1997) 275(5307) 1790-92).

Aberrant activation of Wnt pathway components have been implicated in human tumors. Both Wnt-1 and Wnt2 are up-regulated in NSCLC (You et al., Oncogene (2004) 23(36):6170-4; He et al., Neoplasia (2004) 6(1):7-14) whereas Wnt-7a is down-regulated in most lung cancer cell lines and tumor tissues. Coexpression of both Wnt-7a and the Wnt receptor Frizzled 9 (Fzd9) inhibited cell growth of NCSLC cell lines (Winn et al., J. Biol. Chem. (2005) 280(20): 19625-34). The frizzled receptors Fzd1, Fzd2 and Fzd7 are involved in lung and colon cancers (Sagara et al., Biochem. Biophys. Res. Comm. (1998) 252(1):117-122). Frizzled 3 is involved in lung, cervical and colon cancer, Frizzled 7 in gastric cancer, and Frizzled 10 in gastric and colon cancer (Kirkoshi et al., Int J. Oncol. (2001) 19(4):767-771). The Wnt pathway member Disheveled (DVL) has been shown to be over-expressed in 75% of microdissected NSCLC tissues (Uematsu et al., Oncogene (2003) 22(46): 7218-21).

### SUMMARY

The present disclosure provides compositions, pharmaceutical preparations, kits and methods for inhibiting cell proliferation by contacting a cell expressing Frizzled 8 (Frz8) with a truncated Wnt2 polypeptide which acts as a dominant negative inhibitor of Fzd8 signaling. The present disclosure provides compositions, kits and methods for the detection of cancer by determining the level of Fzd8 and/or Wnt2 expression in a cell.

In one aspect the disclosure provides a dominant negative (dnWnt2) Wnt2 polypeptide comprising an amino acid sequence of a truncated Wnt2 polypeptide comprising five Wnt protein signature motifs, wherein the dnWnt2 polypeptide does not induce Fzd8 signal transduction and is capable of inhibiting induction of Fzd8 signal transduction mediated by a Wnt2 polypeptide. In related embodiments, the dnWnt2 comprises a signal peptide at an N-terminus of the dnWnt2. In further related embodiments, the dnWnt2 polypeptide comprises a heterologous polypeptide (e.g., an eptitope tag) fused to an N- or C-terminal end of the dnWnt2 polypeptide.

The present disclosure also provides nucleic acids comprising a nucleic acid sequence encoding a dnWnt2 polypeptide, vectors comprising such nucleic acid, and host cells comprising such dnWnt2-encoding nucleic acids and/or vectors.

In other aspect, the present disclosure provides methods of inhibiting proliferation of a cancer cell expressing a Frizzled 8 (fzd8) receptor, the method comprising contacting a cancer cell expressing a Fzd8 receptor with a dnWnt2 polypeptide in an amount effective to inhibit Wnt2 polypeptide-mediated Fzd8 signal transduction, wherein said contacting is effective to inhibit Wnt2-mediated cancer cell proliferation. In related embodiments, the cancer cell is of lung, colon, breast, liver, kidney, prostate or skin origin. In further embodiments, the cancer cell is present in a mammal having a Fzd8-expressing cancer.

In another aspect, the present disclosure provides methods of detecting a cancerous cell comprising detecting a level of Frizzled 8 receptor (Fzd8) expression in a test cell, wherein a level of Fzd8 in the test cell that is significantly elevated as compared to a level of Fzd8 expression of a normal cell indicates the test cell is cancerous. In related embodiments, the method involves detecting a level of Wnt2 polypeptide in the cell, wherein a level of Wnt2 in the test cell that is significantly elevated as compared to a level of Wnt2 of a normal cell indicates the test cell is cancerous. In further related embodiments, the test cell is a lung, colon, breast, liver, kidney, prostate or skin cell. In further embodiments, the test cell is a human cell.

In another aspect, the present disclosure provides methods of screening for a candidate agent for activity in inhibiting Frizzled 8 (Fzd8) receptor activation in the presence of a Wnt2 polypeptide comprising contacting a cell that expresses Fzd8 with a candidate agent in the presence of a Wnt2 polypeptide; and detecting Fzd8 signaling in the cell; wherein reduction of Fzd8 signaling in the presence of the candidate agent as compared to Fzd8 signaling in the absence of the candidate agent indicates the candidate agent has activity in inhibiting Wnt2-mediated Fzd8 signal transduction. In related embodiments, the reduction of Fz8 signaling is determined by detecting a level of β-catenin polypeptide in the cell.

In another aspect, the present disclosure provides methods of screening for a candidate agent comprising contacting a Fzd8 receptor with a candidate agent in the presence of a dnWnt2 polypeptide; and assaying the ability of the candidate agent to inhibit binding of the dnWnt2 polypeptide to the Fzd8 receptor; wherein reduction of binding of dnWnt2 polypeptide in the presence of the candidate agent indicates that the candidate agent competes with dnWnt2 polypeptide for binding to the Fzd8 receptor. In related embodiments, the dnWnt2 polypeptide is detectably labeled, and said assaying is by detecting a decrease in bound, detectably labeled dnWnt polypeptide in the presence of the candidate agent. In further related embodiments, the dnWnt2 polypeptide is detectably labeled, and said assaying is by detecting an increase in unbound detectably labeled Wnt2 polypeptide. In further embodiments, contacting is in the presence of a Wnt2 polypeptide. In further related embodiments, the Fzd8 receptor is expressed in a cell, providing a cell-based assay.

Other aspects of the invention will be apparent from the detailed description below.

### BRIEF DESCRIPTION OF THE DRAWINGS

The invention is best understood from the following detailed description when read in conjunction with the accompanying drawings. Included in the drawings are the following figures:

FIG. 1, panel A is graph of results of screening of Wnt2 against ten Frizzled receptors, showing the relative light units (RLU) elicited when Wnt2 specifically binds a Fzd receptor.

FIG. 1, panel B is a graph showing the luciferase activity elicited when cells are transfected with Wnt2 alone, Fzd8 alone or Wnt2 and Fzd8 together.

FIG. 2, panel A is a graph showing the reduction in luciferase activity when cells expressing Fzd8 and Wnt2 are transfected with dominant negative human Wnt2 (dnhWnt2).

FIG. 2, panel B is a Western blot showing β-catenin levels in 3 different cell types when transfected with dnhWnt2 only, a combination of Fzd8 and Wnt2 or a combination of Fzd8, Wnt2 and dnhWnt2.

FIG. 3, panel A is a graph showing the endogenous expression levels of Wnt2 and Fzd8 in various cells.

FIG. 3, panel B is a graph showing cell proliferation of a lung cancer cell line when administered a dnhWnt2.

FIG. 3, panel C is a graph showing cell proliferation of a lung cancer cell line when administered a dnhWnt2.

FIG. 3, panel D a graph showing apoptosis of lung cancer cells transfected with dnhWnt2.

FIG. 4, panel A (top) is a graph showing the number of colonies of cancer cell lines when administered a dnhWnt2. FIG. 4, panel A (bottom) shows the expression of dnhWnt-2 in the cancer cell lines.

FIG. 4, panel B is a graph showing the luciferase activity in cancer cell lines transfected with dominant negative human Wnt2 (dnhWnt2).

FIG. 5, panel A is a graph showing tumor volume in xenograft mouse models.

FIG. 5, panel B is a graph showing tumor mass in xenograft mouse models.

FIG. 5, panel C depicts immunohistochemistry staining of tumor in xenograft mouse models.

FIG. 5, panel D depicts RT-PCR analysis of Wnt target genes.

FIG. 6 is a photograph of Wnt2 and Fzd8 expression levels in isolated primary human lung tumors.

FIG. 7 is a the nucleic acid sequence of human dnWnt2 (SEQ ID NO:1). The signal sequence is encoded by the 75 nucleotides at the 5′ end of SEQ ID NO:1.

FIG. 8 is the polypeptide sequence of human dnWnt2 (SEQ ID NO:2). The signal sequence is included, which is defined by the first 25 amino acids of SEQ ID NO:2. Thus a mature hdnWnt2 is composed of amino acid residues 26

FIG. 9 is an alignment of Wnt2 polypeptides from various species. The Wnt protein signatures are bolded and underlined, and the point of truncation is shown by a downward facing arrow (▾). (human_wnt2—SEQ ID NO:3, chimp_wnt2—SEQ ID NO:4, macaca_wnt2-SEQ ID NO:5, Gorilla_wnt2—SEQ ID NO:6, Baboon_wnt2-SEQ ID NO:7, mouse_wnt2-SEQ ID NO:8, rat_wnt2-SEQ ID NO:9, zebrafish_wnt2-SEQ ID NO:10). The signal sequence is amino acids 1-25 and is shown in bold.

FIG. 10 is the nucleic acid sequence of human Fzd8 (SEQ ID NO:11).

FIG. 11 is the polypeptide sequence of human Fzd8 (SEQ ID NO:12).

### DETAILED DESCRIPTION OF EXEMPLARY EMBODIMENTS

The present disclosure provides compositions, pharmaceutical preparations, kits and methods for inhibiting cell proliferation by contacting a cell expressing Fzd8 with a truncated Wnt2 polypeptide which acts as a dominant negative inhibitor of Fzd8 signaling. The present disclosure provides compositions, kits and methods for the detection of cancer by determining the level of Fzd8 and/or Wnt2 expression in a cell.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the exemplary methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a sample” includes a plurality of such samples and reference to “the molecule” includes reference to one or more molecules and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The term “Wnt polypeptide” refers to a family of polypeptides related by amino acid sequence homology to the Wingless segment polarity genes in Drosophila. “Wnt” is short form of the terms “wingless-related MMTV integration site.” The Wnt polypeptides are from about 38-43 kDa cysteine rich glycoproteins having a signal sequence (Shimizu et al., Cell Growth Diff. (1997) 8(12):1349-1348). The Wnt family of polypeptides comprises at least 19 members, for example, Wnt1, Wnt2, Wnt3, Wnt3A, Wnt4, Wnt5A, Wnt5B, Wnt6, Wnt7A, Wnt7B, Wnt8A, Wnt10A, Wnt10B, Wnt 11, Wnt12, Wnt13, Wnt14, Wnt15, and Wnt16.

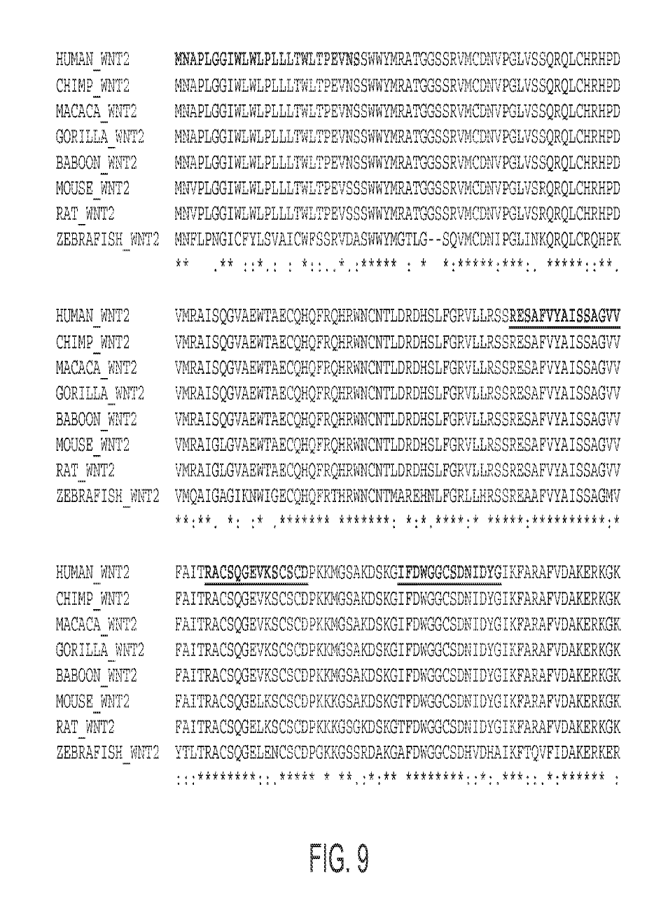

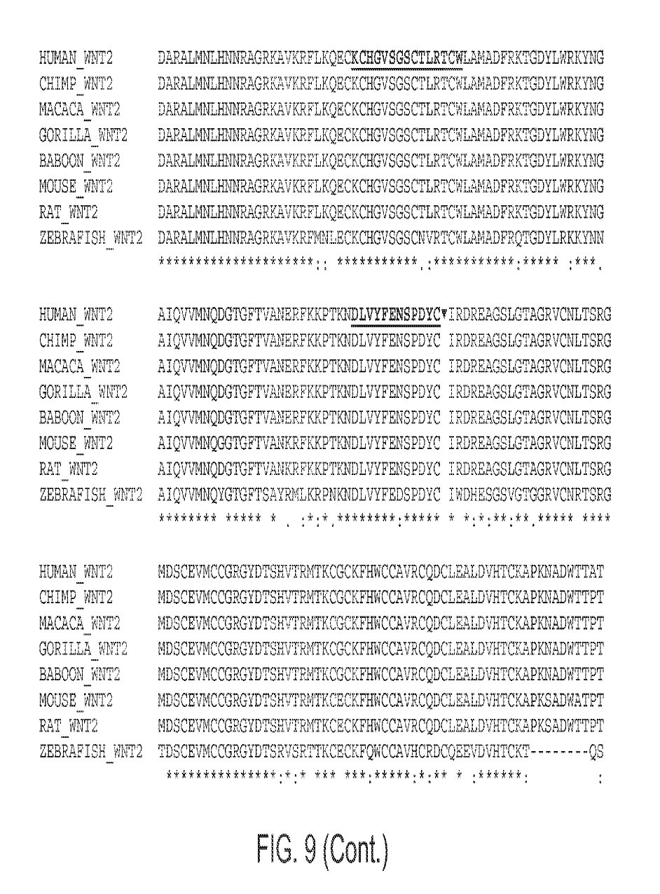

“Wnt2” or “wild-type Wnt2” refers to a full length Wnt2 polypeptide (or, as dictated by the context, its encoding nucleic acid) that interacts with an extracellular domain of a Fzd8 receptor and is capable of activating a Fzd8 receptor to induce signal transduction of the Fzd8 receptor. A Wnt2 is characterized by having 5 Wnt protein signature motifs and at least 90% or greater sequence identity, usually at least 95% or greater amino acid sequence identity, to a contiguous amino acid sequence of a mature Wnt2 polypeptide as illustrated in FIG. 9, particularly to an amino acid sequence of a mature human Wnt2 polypeptide as illustrated in FIG. 9. A Wnt2 polypeptide can be provided in precursor form, as a pro-polypeptide with a signal sequence, as well as in a mature form without a signal sequence. For example, human Wnt2 contains a signal sequence of amino acids 1-25. Exemplary Wnt2 polypeptides and nucleic acids are discussed herein and are available in the art.

The term “Frizzled receptor” as used herein, refers to a family of polypeptides related to the Frizzled gene in Drosophila, which play a role in the development of the fly. The Frizzled family comprises at least 10 members, and is commonly abbreviated “Fzd” in the nomenclature. Examples of mammalian Frizzled genes are as follows; Fzd1, Fzd2, Fzd3, Fzd4, Fzd5, Fzd6, Fzd7, Fzd8, Fzd9 and Fzd10.

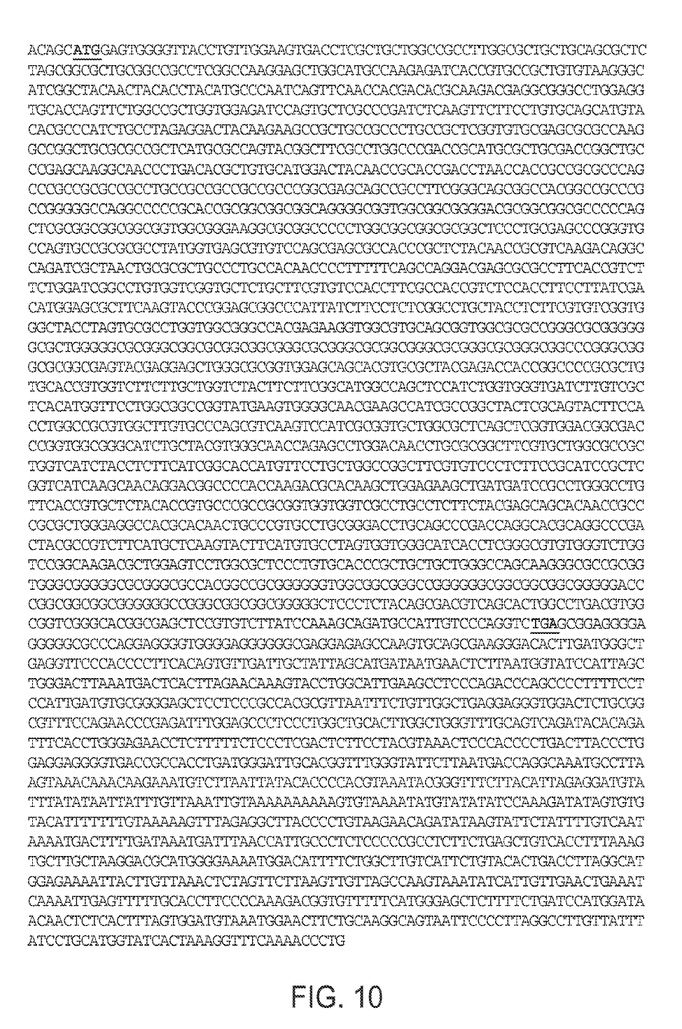

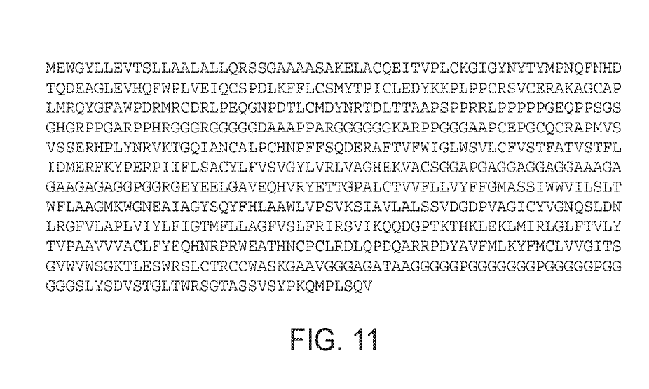

The “Fzd8 receptor” (also referred to herein as “Fzd8”) is a multi-transmembrane polypeptide, with seven membrane spanning regions, ending with the C-terminus inside the cell. The Fzd8 receptor has an N-terminal signal sequence, and a domain of 120 amino acids containing 10 cysteines which comprise a cysteine-rich domain (CRD). (Saitoh et al., Int. J. Oncol. (2001) 18(5):991-996). The nucleotide sequence of Fzd8 is represented in FIG. 10 (SEQ ID NO: 11) with the polypeptide sequence represented in FIG. 11 (SEQ ID NO:12). The Fzd8 sequence has been entered into the NCBI database under Accession number NM_{—}031866.

“Sample” as used herein encompasses samples that may be obtainable from a variety of sources, including (e.g., for assays involving screening of candidate agents) naturally occurring and non-naturally occurring sources. The term “biological sample” encompasses samples obtainable from a source that contains components obtainable from cells, usually mammalian cells, and includes samples obtainable from an individual (e.g., a clinical sample). Biological samples thus include, but are not necessarily limited to cells (including cultured cells), cell supernatants, tissue samples (including solid tissue samples (such as a biopsy specimen), blood samples, and other fluid samples of biological origin), and the like. The definition also includes samples that have been manipulated in any way after their procurement, such as by treatment with reagents; washed; or enriched for certain cell populations, such as cancer cells.

As used herein the term “isolated” is meant to describe a compound of interest (e.g., either a polynucleotide or a polypeptide) that is in an environment different from that in which the compound might naturally occur.

“Purified” as used herein refers to a compound removed from an environment in which it was produced and is at least 60% free, preferably 75% free, and most preferably 90% free from other components with which it is naturally associated or with which it was otherwise associated with during production.

“Mammal” for purposes of treatment refers to any animal classified as a mammal, including humans, non-human primates, and domestic and farm animals, and can include zoo, sports, or pet animals, such as dogs, cats, cattle, horses, sheep, pigs, goats, rabbits, etc. Humans are of particular interest in the context of the treatment methods described herein.

The phrase “mammalian cell” refers to a cell of any mammal as defined above, with human cells being of interest. The phrase refers to cells in vivo, for example, in an organism or in an organ of an organism. The phrase also refers to cells in vitro, for example, cells maintained in cell culture.

The term “cancer” refers to uncontrolled neoplastic cell growth and proliferation, and can be malignant or benign. It can refer to individual cells or tissues comprised of multiple cell types.

The phrases “operably linked” refer to functionally related nucleic acid sequences. By way of example, a regulatory sequence is operably linked with a nucleic acid sequence encoding a polypeptide if the regulatory sequence can exert a positive or negative effect on the expression of the encoded polypeptide. While operably linked nucleic acid sequences can be contiguous with the nucleic acid sequence that they control, the phrase “operably linked” is not meant to be limited to those situations in which the regulatory sequences are contiguous with the nucleic acid sequences they control.

The term “candidate agent” is meant to encompass any agent that is suitable for screening for activity in modulating or mimicking a biological activity of interest (e.g., a Fzd8 receptor activity, activity of a dnWnt2 peptide, and the like), and can include any substance, molecule, element, compound, entity, or a combination thereof. It includes, but is not limited to, e.g., proteins (including antibodies), oligopeptides, small organic molecules, and the like. It can be a natural product, a synthetic compound, or a chemical compound, or a combination of two or more compounds.

The phrase “effective amount” in the context of a dnWnt2 peptide described herein, refers to an amount of a dnWnt2 polypeptide effective to cause a decrease in Fzd8 signal transduction in the presence of a Fzd8 activating agent, e.g., a wild-type Wnt2 polypeptide. Fzd8 signal transduction is considered to be decreased when, in the presence of a wild-type Wnt2 polypeptide, Fzd8 signaling in a cell is decreased in the presence of a dnWnt2 as compared to Fzd8 signaling in a cell in the presence of a wild-type Wnt2 peptide. Of particular interest is an amount effective to provide for at least about 45% or more decrease in Fzd8 signal transduction in the presence of wild-type Wnt2, including about 50% or more, about 60% or more, about 70% or more, about 75% or more, about 80% or more, about 90% or more decrease in Fzd8 signaling activity in a cell contacted with dnWnt2 in the presence of wild-type Wnt2 compared to the absence of a dnWnt2 peptide.

The term “contacting” refers to combining at least two components (e.g. two polypeptides, a polypeptide and an antibody or an antibody and a cell). The contacting can occur in vitro (e.g., substances combined in a test tube) or in vivo (e.g., a dnWnt2 contacting a tumor cell in the lung).

The term “sequence identity” refers to a number of nucleotides that are identically shared between a query nucleotide sequence and a reference nucleotide sequence. Sequence identity can also refer to the number of amino acid residues that are identically shared between a query amino acid sequence and a reference amino acid sequence. The first step in determining sequence identity is to align the two sequences and introduce gaps if necessary to achieve the maximum alignment. Conservative substitutions, for example, alanine matching to valine, are not considered in determining sequence identity between amino acid sequences. The alignment can be global, spanning the full length of the entire sequence. Alternatively, the alignment can be local, over specific domains. Next, the total number of identical matches is determined. Sequence identity is calculated by dividing this total by the number of nucleotides or amino acids in the maximum alignment. For example, if two amino acid sequences, each 50 amino acids in length are aligned and there are 5 differences between them, then this equals 45 total identical matches. This total is divided by the number (50) in the maximum alignment, so 45/50 equals 90% sequence identity. A reference sequence will be at least about 5 amino acids (or 15 nucleotides) in length, at least about 10 amino acids (or 30 nucleotides) in length, and at least about 15 amino acids (or 45 nucleotides) in length. Computer based algorithms such as BLAST, BLAST-2 and DNASTAR™ are known in the art for determining sequence identity. (Altschul et al., J. Mol. Biol., (1990) 215:403-410)

The term “heterologous” is meant that a first entity and second entity are provided in an association that is not normally found in nature. For example, a “heterologous polypeptide” contains an amino acid sequence not found in nature, e.g., due to the fusion between an amino acid sequence of a first protein and an amino acid sequence of a second protein.

The term “recombinant” as used herein, refers to a cell that replicates a heterologous nucleic acid or expresses a polypeptide encoded by the heterologous nucleic acid, as well as to expression products from such cells and nucleic acid encoding such expression products (e.g., “recombinant polypeptide”, “recombinant nucleic acid”).

The term “vector” is used to describe a vehicle that is capable of incorporating at least one heterologous sequence and transferring the heterologous sequence to a host cell.

The term “label” as used herein refers to a compound which is directly detectable (e.g. a fluorescent tag) or indirectly detectable (e.g., produces a chemical or enzymatic change in a substrate compound which is detectable, capable of being bound by a directly detectable labeled agent (e.g., detectably labeled antibody), and the like.

The term “immunoadhesin” as used herein refers to a heterologous polypeptide composed of a polypeptide having a desired characteristic (e.g., such as that of a dnWnt2 peptide) fused to an immunoglobulin constant domain sequence. The immunoglobulin constant domain sequence can be selected from any immunoglobulin, for example, IgG1 or subtypes thereof, IgA or subtypes thereof, IgE or subtypes thereof, IgM or subtypes thereof, or IgD or subtypes thereof.

### Overview

The present disclosure provides compositions, pharmaceutical preparations, kits and methods for inhibiting cell proliferation by contacting a cell expressing Fzd8 with a truncated Wnt2 polypeptide which acts as a dominant negative inhibitor of Fzd8 signal transduction. The present disclosure provides compositions, kits and methods for the detection of cancer by determining the level of Fzd8 and/or Wnt2 expression in a cell.

### Compositions

The present disclosure provides for dnWnt2 polypeptides, as well as nucleic acids encoding such. Exemplary dnWnt2 polypeptides and encoding nucleic acids are described below.

dnWnt2 Polypeptides

As used herein the term “dominant negative Wnt2” or “dnWnt2” refers to a truncated Wnt2 polypeptide that is capable of inhibiting Fzd8 signal transduction in the presence of a full length Wnt2 polypeptide. “dnWnt2 polypeptide” and “dnWnt2 peptide” are used herein interchangeably. A dnWnt2 can be any truncated Wnt2 that comprises from N-terminus to C-terminus, 5 Wnt protein signature motifs, does not induce detectable or significant Fzd8 signal transduction and is capable of inhibiting induction of Fzd8 signal transduction in the presence of Wnt2 polypeptide. The dnWnt2 can be based on the amino acid sequences from any species, for example, human, as shown in FIG. 8 (SEQ ID NO:2). dnWnt2 having an amino acid sequence derived from a human Wnt2 polypeptide, referred to herein as a: “human dnWnt2” or “dnhWnt2”, and are of particular interest. dnhWnt2 is a truncated human Wnt2 comprising from N-terminus to C-terminus, 5 Wnt protein signature motifs which are derived from a human wild-type Wnt2, does not induce Fzd8 signal transduction, and is capable of inhibiting induction of Fzd8 signal transduction in the presence of Wnt2 polypeptide.

dnWnt2 polypeptides are composed of an amino acid sequence of a truncated Wnt2 polypeptide and certain Wnt polypeptide motifs. These are referred to herein as “Wnt protein signature motifs”, and are referred to as motifs 1-5, numbered from N- to C-terminus of the amino acid sequence. The consensus sequence of the Wnt protein signature motifs 1-5 are:

(SEQ ID NO: 13)
1. (RE (A/T/S)AF(T/V/I)(F/Y/H)AI(S/T/L)SA(G/A)
(V/M)(A/V/T))
(SEQ ID NO: 14)
2. (R(A/S/D)C(S/A)(Q/E/R)G(E/A/S/I)(L/V/I/S)(E/K)
(N/S/R)C(S/T/G)CD)
(SEQ ID NO: 15)
3. (I/T/A)(W/F)(H/S/D/Q)W(G/Q)GC(S/G)D(H/N)(V/I)
(K/E/D)(F/Y/H)(G/A)
(SEQ ID NO: 16)
4. (KCHG(M/V)SG(S/R)C(T/N/E)(V/L/M)(R/K)TCW)
(SEQ ID NO: 17)
5. (DLV(Y/F)(F/M)(E/D/T)(N/D/K/P)SP(D/N)(Y/F)C)

The exemplary Wnt2 polypeptides and exemplary Wnt protein signature motifs are set out in FIG. 9, where the Wnt protein signature motifs are set out bold and underlined text. For example, the human dnWnt2 is composed of 1-278 amino acids and contains 5 Wnt protein signature motifs; located at amino acids 106-120, 125-138, 152-164, 207-221, and 267-278.

In general, dnWnt2 polypeptides include those having five Wnt protein signature motifs of about 90%, about 95%, or greater amino acid sequence identity to a contiguous amino acid sequence of the contiguous Wnt protein signature motifs of FIG. 9, over a contiguous amino acid sequence of a mature Wnt2 (e.g., a mature human Wnt2) exemplified in FIG. 9, and/or over a contiguous amino acid sequence of an immature Wnt2 (e.g., a precursor polypeptide of a human Wnt2) exemplified in FIG. 9. The amino acid sequence intervening the Wnt protein signature motifs can be of about 90%, about 95%, or greater amino acid sequence identity to a contiguous amino acid sequence as exemplified in FIG. 9. For example, a dnWnt2 polypeptide including the five Wnt protein signature motifs and the amino acid sequences intervening the Wnt protein signature motifs provides for a dnWnt2 polypeptide having about 90%, about 95%, or greater amino acid sequence identity of a contiguous amino acid sequence of amino acids 1-278, or of amino acid residues 26-278 for a dnWnt2 without a signal sequence, of a Wnt2 polypeptide exemplified in FIG. 9, with a dnWnt2 having such amino acid sequence identity to a contiguous amino acid sequence of a human Wnt2 polypeptide being of particular interest. The alignments of FIG. 9 provides guidance to one of skill in the art as to where amino acid changes could be made in one or both of the 5 Wnt protein signature motifs and/or intervening amino acid sequences without loss of relevant function in inhibiting Fzd8 activation in the presence of wild-type Wnt2.

In one example, following the amino acid sequence of the fifth Wnt protein signature motif, a dnWnt2 lacks an amino acid sequence of a wild-type Wnt2, e.g., of a wild-type human Wnt2. Stated differently, a dnWnt2 lacks about 70 to 85 amino acids (e.g., 82 amino acids) of the C-terminus of a wild-type Wnt2. In the exemplary dnWnt2 polypeptides described, the point of truncation is shown in FIG. 9, by a downward arrow (▾), providing for a C-terminal deletion of 82 amino acid residues relative to a wild-type human Wnt2.

dnWnt2 can be provided in precursor form, (a pro-polypeptide) as well as a mature form. For example, human dnWnt2 in pro-polypeptide form includes a signal peptide, predicted to be from amino acids 1-25, and can be cleaved during processing of the polypeptide. Thus, in the human dnWnt2 example, amino acids 1-25 are not present in the mature form. This is represented in FIG. 9, with the signal sequence highlighted in bold text.

Mature dnWnt2 polypeptides (i.e., dnWnt2 polypeptides lacking the signal sequence) are usually at least 250 amino acid residues in length, but are shorter than full-length, mature Wnt2 polypeptide, e.g., less than 335 amino acids, less than 300 amino acids, to less than 275 amino acids, to about 250 amino acid residues in length (e.g., 253 amino acid residues in length).

The sequences of a number of Wnt2 polypeptides have been described, and can provide guidance for production of a dnWnt2 of the present disclosure. For example, the amino acid sequence of human Wnt2 (GenBank accession no. P09544), Chimp Wnt2 (GenBank accession no. XP_{—}519328), Macaca Wnt2 (GenBank accession no. AAY89005), Colobus monkey Wnt2 (GenBank accession no. Q07DY7), Baboon Wnt2 (GenBank accession no. A0M8S1), Gibbon Wnt2 (GenBank accession no. Q07DX7), Green monkey Wnt2 (GenBank accession no. Q2IBB0), Orangutan Wnt2 (GenBank accession no. Q21BE2), Gorilla Wnt2 (GenBank accession no. ABC87453), Lemur Wnt2 (GenBank accession no. ABC87442), mouse Wnt2 (GenBank accession no. NM_{—}023653), rat Wnt2 (GenBank accession no. AAR16313), Bovine Wnt2 (GenBank accession no. NM_{—}001013001), Cat Wnt2 (GenBank accession no. A0M8T2), Hedgehog Wnt2 (GenBank accession no. A1×153), Opossum Wnt2 (GenBank accession no. Q2QL96), Rabbit Wnt2 (GenBank accession no. Q09YN1), Chicken Wnt2 (GenBank accession no. AY753287), Elephant Wnt2 (GenBank accession no. Q108U2), Horse Wnt2 (GenBank accession no. Q2QLA5), Zebrafish Wnt2 (GenBank accession no. NP_{—}571025), Fugu Wnt2 (GenBank accession no. AAL40368), Sea Anemone Wnt2 (GenBank accession no. AAW28132), Xenopus Wnt2 (GenBank accession no. NP_{—}001081637) and Drosophila Wnt2 (GenBank accession no. P28465) are known in the art.

dnWnt2 fusion polypeptides are also provided, in which a dnWnt2 amino acid sequence is joined to a heterologous amino acid sequence. Exemplary dnWnt2 fusion polypeptides include those comprising a dnWnt2 fused to an “epitope tag” polypeptide which can be readily detectable by an anti-tag antibody. The epitope tag can be placed at any suitable position relative to the dnWnt2 (e.g., at the N-terminus or C-terminus), usually so that the epitope tag does not interfere with activity of dnWnt2 in inhibiting Fzd8 activation as described herein. The epitope tag allows the fusion polypeptide to be readily detected, or readily purified by affinity purification using an antibody or an immobilized affinity molecule that binds to the epitope tag. Examples of epitope tags known in the art are as follows: poly-histidine (poly-his) tags; the influenza HA tag (Field et al., Mol. Cell. Biol., (1988) 8:2159-2165; the c-myc tag (Evan et al., Mol. Cell. Biol., (1985) 5:3610-3616; Herpes Simplex virus glycoprotein D (gD) tag (Paborsky et al., Prot. Eng., (1990) 3(6):547-553); the Flag-peptide (Hopp et al., BioTech., (1988) 6:1204-1210); the KT3 epitope peptide (Martin et al., Science, (1992)255:192-194); the α-tubulin epitope peptide (Skinner et al., J. Biol. Chem., (1991) 266:15163-15166; and the T7 gene 10 protein peptide tag (Lutz-Freyermuth et al., PNAS USA, (1990)87:6393-6397.

Further exemplary dnWnt2 fusion polypeptides include fusion of a dnWnt2 peptide with a heterologous polypeptide that can provide for a desired characteristic, such as increased serum half-life, resistance to proteolysis, delivery to a desired cell (e.g., a cell expressing a Fzd8 receptor), and the like. In one example, the heterologous polypeptide is an immunoglobulin or a fragment thereof. For example, dnWnt2 immunoadhesins are provided as a fusion of a dnWnt2 to an Fc portion of an antibody (e.g., an IgG). The immunoadhesin can include the hinge, CH₁, CH₂ and optionally the CH₃ regions of an IgG1 molecule. The production of immunoadhesins using a binding region of interest can be accomplished according to methods known in the art (see, e.g., U.S. Pat. No. 5,428,130).

dnWnt polypeptides can be made by recombinant methods (e.g., by expression for a recombinant mammalian host cell). Alternatively, dnWnt2 peptides can be made by synthetic methods known in the art.

dnWnt2-Encoding Nucleic Acids

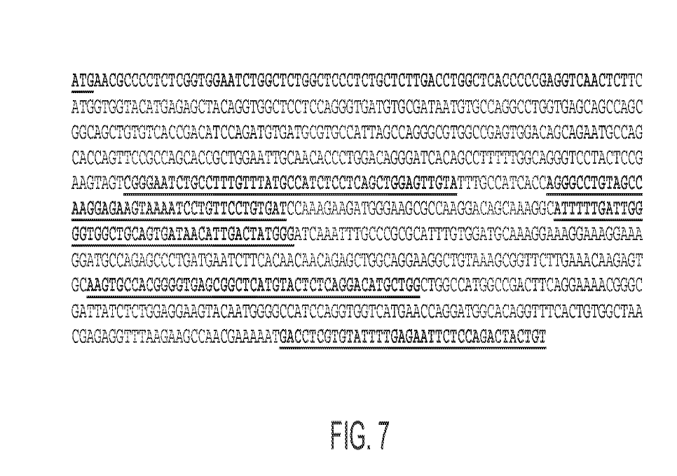

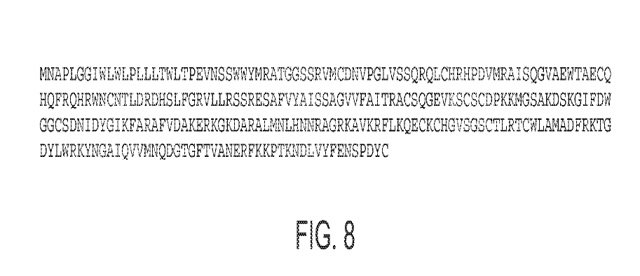

For all dnWnt2 polypeptide described herein, nucleic acids encoding such dnWnt2 are likewise contemplated by the present disclosure. The nucleic acid sequences of the dnWnt2 polypeptides exemplified herein can be readily ascertained based on the amino acid sequences provided herein as well as those known in the art, and the knowledge of the genetic code. An exemplary nucleic acid encoding a dnWnt2 having an amino acid sequence derived from a human Wnt2 is shown in FIG. 7 (SEQ ID NO:1). The start codon (ATG) is bolded and underlined. The five Wnt protein signature motifs are also bolded and underlined. In addition, an exemplary nucleic acid sequence encoding the optional signal peptide is indicated at nucleotides 1-75 of FIG. 7 and are shown as bolded, but not underlined text. Nucleic acids encoding dnWnt2 fusion polypeptides as described herein are also contemplated.

In addition, the sequences of a number of Wnt2 nucleic acids have been described. For example, the nucleic acid sequence of human Wnt2 (GenBank accession no. X07876), Chimp Wnt2 (GenBank accession no. XM_{—}519328), Macaca Wnt2 (GenBank accession no. XM_{—}001102782), mouse Wnt2 (GenBank accession no. NM_{—}023653), rat Wnt2 (GenBank accession no. XM_{—}001059030), bovine Wnt2 (GenBank accession no. NM_{—}001013001), Zebrafish Wnt2 (GenBank accession no. NM_{—}130950), Xenopus Wnt2 (GenBank accession no. NP_{—}001081637) and Drosophila Wnt2 (GenBank accession no. NM_{—}057462) are known in the art.

The present disclosure provides for nucleic acids encoding a dnWnt2 fusion polypeptide. The nucleic acids encoding dnWnt2 can be ligated to nucleic acids encoding an epitope tag to produce a fusion nucleic acid encoding the desired fusion polypeptide. For example, a dnWnt2 coding region can be ligated to a nucleic acid encoding the c-myc tag. In addition, the nucleic acids can encode an immunoglobulin fusion polypeptide. For example, nucleic acid encoding human dnWnt2 can be ligated to nucleic acid encoding the hinge, CH', CH² and optionally the CH³ regions of an IgG molecule.

The present disclosure provides for a dnWnt2 sequence containing variations. For example, a nucleic acid encoding a truncated dnWnt2 polypeptide can have 90%, 95% or greater amino acid sequence identity across each of the contiguous Wnt protein signature motifs, over a contiguous amino acid sequence of a mature Wnt2 (e.g., a mature human Wnt2), and/or over a contiguous amino acid sequence of an immature Wnt2 (e.g., a precursor polypeptide of a human Wnt2). Substitutions, insertions, and deletions in the nucleic acid sequence can be made based on guidance from the amino acid alignment provided in FIG. 9 as well as the knowledge of the genetic code (and its inherent degeneracy).

The present disclosure provides for nucleic acids with changes from individual to individual. These are referred to herein as “allelic variants.” It will be appreciated by one of skill in the art that allelic variants can change the sequence of the encoded polypeptide or be “silent mutations,” where the change in the nucleic acid sequence does not result in a change in the encoded polypeptide.

Vectors Containing dnWnt2 Nucleic Acid

The present disclosure provides for a vector comprising a Wnt2 nucleic acid. The vector can be a plasmid, cosmid, yeast artificial chromosome (YAC), bacterial artificial chromosome (BAC), viral vector or bacteriophage. The vectors can provide for replication of dnWnt2 nucleic acids, expression of dnWnt2 polypeptides or integration of dnWnt2 nucleic acids into the chromosome of a host cell. The choice of vector is dependent on the desired purpose. Certain cloning vectors are useful for cloning, mutation and manipulation of the dnWnt2 nucleic acid. Other vectors are useful for expression of the dnWnt2 polypeptide, being able to express the polypeptide in large amounts for purification purposes or to express the dnWnt2 polypeptide in a temporal or tissue specific manner, for example, expression of dnWnt2 only in lung cells. The vector can also be chosen on the basis of the host cell, e.g., to facilitate expression in bacteria, mammalian cells, insect cells, fish cell (e.g., zebrafish) and/or amphibian cells. The choice of matching vector to host cell is apparent to one of skill in the art, and the types of host cells are discussed below. Many vectors or vector systems are available commercially, for example, the pET bacterial expression system (Invitrogen®, Carlsbad Calif.).

Vectors can include various components including, but not limited to, an origin of replication, one or more marker or selectable genes (e.g. GFP, neo), promoters, enhancers, terminators, poly-adenylation sequences, repressors or activators. Such elements are provided in the vector so as to be operably linked to the coding region of the dnWnt2-encoding nucleic acid, thereby facilitating expression in a host cell of interest. Cloning and expression vectors can contain an origin of replication which allows the vector to replicate in the host cells. Vectors can also include a selectable marker, e.g., to confer a resistance to a drug or compliment deficiencies in growth. Examples of drug resistance markers include, but are not limited to, ampicillin, tetracycline, neomycin or methotrexate. Examples of other marker genes can be the fluorescent polypeptides such one of the members of the fluorescent family of proteins, for example, GFP, YFP, BFP, RFP etc. These markers can be contained on the same vector as the gene of interest or can be on separate vectors and co-transfected with the vector containing the gene of interest.

The vector can contain a promoter that is suitable for expression of the dnWnt2 in mammalian cells, which promoter can be operably linked to provide for inducible or constitutive expression of a dnWnt2 peptide. Exemplary inducible promoters include, for example, the metallothionine promoter or an ecdysone-responsive promoter. Exemplary constitutive promoters include, for example, the viral promoters from cytomegalovirus (CMV), Rous Sarcoma virus (RSV), Simian virus 40 (SV40), avian sarcoma virus, the β-actin promoter and the heat-shock promoters. The promoter can be chosen for its tissue specificity. Certain promoters only express in certain tissues, and when it is desirable to express the polypeptide of interest only in a selected tissue, one of these promoters can be used. For example, a fragment of the lung surfactant −C promoter was used in a lentiviral vector system to express GFP only in lung epithelial cells (Wunderlich et al., Hum. Gene Ther. (2008) 19(1):39-52). The choice of promoter will be apparent to one of skill in the art for the desired host cell system.

The vector encoding dnWnt2 can be a viral vector. Examples of viral vectors include retroviral vectors, such as: adenovirus, simian virus 40 (SV40), cytomegalovirus (CMV), Moloney murine leukemia virus (MoMuLv), Rous Sarcoma Virus (RSV), lentivirus, herpesvirus, poxvirus and vaccinia virus. A viral vector can be used to facilitate expression in a target cell, e.g., for production of dnWnt2 or for use in therapy (e.g., to deliver dnWnt2 to a patient by expression from the vector). Where used for therapy, dnWnt2-encoding vectors (e.g, viral vectors), can be administered directly to the patient via an appropriate route or can be administered using an ex vivo strategy using patient cells (autologous) or allogeneic cells, which are suitable for administration to the patient to be treated.

Host Cells Containing Wnt

Host cells modified to provide for expression of a dnWnt2 peptide disclosed herein are also contemplated. Such host cells can be modified to express a dnWnt2 peptide from either an episomal or genomically integrated nucleic acid. Such host cells can be produced by any suitable method, e.g., electroporation, transfection or transformation with a vector encoding a dnWnt2 peptide. Host cells can be selected according to a desired use (e.g., mammalian cell expression), and modified to provide for dnWnt2 expression according to methods well known in the art. Techniques for introducing the vectors into host cells and subsequent culture of the host cells are well known in the art.

Host cells (e.g., mammalian host cells) suitable for replication and expression of dnWnt2 containing vectors are provided, wherein the cells may be stably or transiently transfected and/or stably or transiently express a dnWnt2. Such dnWnt2-expressing mammalian cells find use in, for example, production of a dnWnt2 polypeptide. Production of dnWnt2 in mammalian cells can provide for post-translational modifications of dnWnt2 and/or heterologous amino acids to which it may be fused (e.g., glycosylation, cleavage of signal peptide (if present)). In addition, mammalian cell lines can be selected for use in replicating, packaging and producing high titers of virus particles which contain a dnWnt2 of interest or nucleic acid-encoding dnWnt2. Such dnWnt2 containing viruses can then be used to provide for delivery of dnWnt2-encoding nucleic acids and dnWnt2 peptides.

Exemplary host cells include bacteria, yeast, mammalian cells (e.g., human cells or cell lines), insect cells, and the like. Examples of bacterial host cells include E. coli and other bacteria which can find use in cloning, manipulation and production of dnWnt2 nucleic acids or the production of dnWnt2 polypeptide. Examples of mammalian cells include, but are not limited to, Chinese hamster ovary (CHO) cells, HEK 293 cells, human cervical carcinoma cells (Hela), canine kidney cells (MDCK), human liver cells (HepG2), baby hamster kidney cells (BHK), and monkey kidney cells (CV1).

Pharmaceutical Preparations Containing dnWnt2 Peptides

The present disclosure also provides pharmaceutical compositions containing a dnWnt2 peptide as disclosed herein. In general, such compositions include a dnWnt2 peptide and a suitable pharmaceutically acceptable carrier. The phrase “pharmaceutically acceptable carrier” refers to compositions that facilitate the delivery of dnWnt2 to a cell expressing Fzd8 and includes, but is not limited to, solvents or dispersants, coatings, isotonic agents, agents that mediate absorption time or release of the dnWnt2, and the like. Formulations suitable for bolus delivery of dnWnt2 are contemplated, as are sustained release formulations to provide depot injections (e.g., implants).

The methods for preparing pharmaceutical compositions of the invention will be known to those skilled in the art and are described in Remington's Pharmaceutical Sciences, Mack Publishing Company, Easton, Pa., 17th edition, 1985; Remington: The Science and Practice of Pharmacy, A. R. Gennaro, (2000) Lippincott, Williams & Wilkins. The composition or formulation to be administered will, in any event, contain a quantity of the agent adequate to achieve a desired effect in a subject that can be described as a clinical benefit in a dnWnt2-based therapy-responsive patient. A dosage of the dnWnt2 can be about 0.1 to 100 mg/kg of body weight per day. If the dnWnt2 is administered to a body cavity or into an organ, the dose range can be adjusted lower or higher depending on the response.

The dnWnt2 of the invention can be administered by a medically acceptable route, depending on the cancer conditions or the location of the cancer. Administration routes include injection, for example the injection can be subcutaneous, intravenous, intramuscular or intravascular etc. Administration can include inhalation, oral, nasal, rectal, or ophthalmic routes.

For a predisposition to Fzd8-expressing cancer, a dnWnt2 can be administered on a prophylactic basis. An effective amount of dnWnt2 that will prevent or slow the progression of cancer is known as a “prophylactic effective dose.” The prophylactic effective dose will depend on the factors of weight, age, administration route, and seriousness of the predisposition. The dose can be lower or the same as the effective dose used in treating diagnosed cancer.

### Methods of Inhibiting Cancer Cell Proliferation

The present disclosure provides methods of inhibiting cancer cell proliferation by exposing a Fzd8-expressing cancerous cell to a dnWnt2 peptide. In general, a dnWnt2 peptide is administered in such a manner so that it contacts the Fzd8 receptor of cancerous cells in a subject so as to inhibit Wnt2-mediated proliferation. The dnWnt2 peptide can be administered systemically or, particularly where the cancerous cells are present in a solid or semi-solid tumor, locally to an appropriate site, for example, injection directly into a tumor or a tumor bed. dnWnt2 peptides can also be administered indirectly, for example, by introducing a vector expressing dnWnt2 into a cell expressing Fzd8 or into cells that are not expressing Fzd8, but are adjacent cells expressing Fzd8, and which secrete the dnWnt2 peptide into the local microenvironment, thereby delivering dnWnt2 peptide to the Fzd8-expressing cancerous cells. In general, the cancers particularly amenable to therapy are those that express a Fzd8 receptor and that are exposed to Wnt2 polypeptide (e.g., by expression of Wnt2 by the cell expressing the Fzd8 receptor or by expression of Wnt2 elsewhere in the subject, resulting in exposure of the Fzd8-expressing cancerous cells to Wnt2).

A reduction in cell proliferation is considered to provide a clinical benefit when the proliferation of Fzd8 expressing cells contacted with dnWnt2 is lower than proliferation of Fzd8 expressing cells contacted with wild type Wnt2. In certain instances, cell proliferation will be reduced by about 30%, reduced by about 35%, reduced by about 40%, reduced by about 45%, reduced by about 50%, reduced by about 55%, reduced by about 60%, reduced by about 65%, reduced by about 70%, reduced by about 75%, reduced by about 80%, reduced by about 85%, or reduced by 90% or greater. Reduction in cell proliferation can also be inferred by, for example, reduction in or maintenance of tumor size, reduction in or maintenance of tumor volume, reduction in or maintenance of metabolic activity of tumor cells, and the like. Reduction in tumor load (e.g., as assessed by, for example, reduction in tumor size, volume, and the like) is also of clinical benefit to the patient. Arresting growth of the tumor (referred to as tumor “maintenance” or “disease control”) is also considered clinically beneficial to the patient. Assessment of responsiveness to therapy can be accomplished by histology, particularly where the tumor is not a solid tumor.

An effective amount of a dnWnt2 to be administered to a subject in need of treatment can be readily determined on a case-by-case basis. Factors to be considered when determining an effective amount involve body weight, age, stage of the cancer, location of the cancerous cells, duration of the treatment, response to the initial treatment and if the administration of dnWnt2 is performed in combination with surgery, chemotherapy, radiation therapy, and the like. The effectiveness of dnWnt2-based therapy can be assessed by, for example, monitoring cell proliferation in a patient, e.g., by measuring tumor size, tumor volume, and/or metabolic activity of tumor cells. Tumor responsiveness can be monitored by means known in the art such as X-ray, MRI, or biopsy of the tumor followed by histological analysis.

Cancers amenable to dnWnt2-based therapy as disclosed herein include any cancer that expresses a cell surface Fzd8 receptor and for which proliferation is mediated at least in part by Wnt2-mediated inducement of Fzd8 signal transduction. Such cancers can be identified by, for example, assessing Fzd8 expression and, optionally, Wnt2 expression. Fzd8-expressing cancerous cells can be of lung, colon, breast, liver, kidney, prostate or skin (e.g., melanoma) origin. For example, Fzd8-expressing lung cancers amenable to dnWnt2-based therapy include, but are not necessarily limited to, non-small cell lung carcinoma (NSCLC), small cell lung cancer (SCLC), adenocarcinoma, squamous cell carcinoma and large cell carcinoma.

Combination Therapy Using the Compounds of the Invention

The dnWnt2 polypeptides described herein may be formulated with, or otherwise administered in combination with, other pharmaceutically active agents, including other agents that activate or suppress a biochemical activity of a cancerous cell, such as a chemotherapeutic agent. Accordingly, dnWnt2 peptides may find use in enhancing the effectiveness of another chemotherapeutic. Moreover, dnWnt2 polypeptides may be used as delivery agents to facilitate delivery of a chemotherapeutic agent to a Fzd8-expressing cell.

Accordingly, the present disclosure provides for combination therapy by conjugating an anti-proliferative agent directly to dnWnt2. Such anti-proliferative agents can be toxins, for example, ricin A chain, maytansinoids, bacterial toxins (e.g., gelonin, saporin, sarcin, aspergillin, diphtheria toxin and pseudomonas toxin). Alternatively, anti-proliferative agents can be anti-tubulin compounds, for example, taxol, colchicine, vinblastine, vincristine, vindescine and combretastatins. Alternatively, anti-proliferative agents can be chemotherapeutic agents. For example, doxorubicin, methotrexate, daunomycin, neocarzinostatin, trenimon and macromycin. The anti-proliferative agents can be conjugated directly to the dnWnt2 or through a linker molecule. The linker molecule can be cleavable or non-cleavable.

The anti-proliferative agent can be a highly radioactive isotope. For example the dnWnt2 can be synthesized in the presence of radioactivity which will incorporate itself into the dnWnt2. Alternatively, the radioactive isotope can be directly conjugated to the dnWnt2 polypeptide. Radioactive isotopes are commonly used in the treatment of cancer and examples include, but are not limited to, I¹²⁵, I¹³¹, I¹²³, Y⁹⁰, P³², At²¹¹, Re¹⁸⁶, Re¹⁸⁸, Sm¹⁵³, Bi²¹², Pb²¹². For example, Isotopes such as I¹²³ and Re¹⁸⁶ can be attached to the dnWnt2 via the cysteine residues and Yttrium-90 (Y⁹⁰) can be attached to the lysine residues.

Alternatively, the present disclosure provides for combination therapy when the dnWnt2 is administered in parallel with another agent. Examples of chemotherapeutic agents for use in combination therapy with dnWnt2 include, but are not limited to, daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES).

dnWnt2 peptides can also be administered in conjunction with an RNAi molecule(s), e.g., which provides for a decrease in expression of Wnt2, Fzd8, or both in a cancerous cells. RNAi refers to interfering RNAs, which reduce or “knock down” gene expression (Fire et al., Nature (1998) 391:806-811). A specific class of RNAi is short inhibitory RNAs or siRNAs. The design and administration of siRNAs to knock down gene expression is well known in the art and can be found, for example, in US published application 2006/0040883. siRNAs are less than 100 nucleotides, and can be between 5 and 100 nucleotides.

The compounds described herein for use in combination therapy with a dnWnt2 may be administered by the same route of administration (e.g. intrapulmonary, oral, enteral, etc.) that the compounds are administered. In the alternative, the compounds for use in combination therapy with a dnWnt2 may be administered by a different route of administration that the compounds are administered.

### Methods of Detecting Cancer

The Examples of the present disclosure also find that Fzd8 and Wnt2 are expressed in cancerous cells. As such, assaying for Fzd8 expression or Fzd8 activity, which optionally can be combined with analysis of Wnt2 expression, can be used in facilitating detection of cancerous cells. Such methods can find use in facilitating a diagnosis of cancer in a subject (e.g., a human subject). Such methods can also find use in identifying a subject having a Fzd8-expressing cancer which may be amenable to therapy using a dnWnt2 peptide as disclosed herein, thereby guiding treatment decisions by a clinician or other healthcare provider.

Detection of Fzd8 expression (and detection of Wnt2 expression) in a cell can be accomplished in a variety of ways. Exemplary methods are described below.

Detecting Fzd8/Wnt2 Expression in a Cell

The present disclosure provides for a method of detecting expression of Fzd8 in a cell. A level of Fzd8 expression can be accomplished by a variety of methods known in the art. For example, mRNA-encoding Fzd8 can be detected using Northern blotting, dot blotting, microarray analysis, in situ hybridization, or nucleic acid amplification methods (e.g., reverse-transcriptase PCR (RT-PCR), e.g., quantitative RT-PCR).

Assaying Fzd8 expression using nucleic acid amplification and/or microarray methods to detect Fzd8-encoding RNA transcripts (e.g., PCR, especially RT-PCR) is of particular interest. Probes and primers can be readily designed using the knowledge in the art, which includes the sequences of Fzd8-encoding nucleic acid sequences. For example, the nucleic acid sequence of human Fzd8 is found in FIG. 10 and available at GenBank accession number (NM_{—}031866). Microarray analysis can be performed by anchoring Fzd8 specific probes to a solid support (e.g., a gene chip) then conducting hybridization and detection of labeled Fzd8 mRNA (see U.S. Pat. No. 6,505,125). The level of the Fzd8 expression in a test cell is generally compared to a level of Fzd8 expression in a control cell, where the control cell may optionally be run in a parallel sample. In some embodiments, a sample suspected of containing cancerous cells of a subject is assayed in parallel with normal cells. The cancerous cells and the normal cells may be obtained from the same patient, e.g., to provide an internal negative control. A level of Fzd8 expression that is significantly higher in the test cell compared to a level of Fzd expression of a normal cell indicates the test cell is, or has a propensity to become, a cancerous cell. In some embodiments, the cancer is other than a liver cancer.

In addition to assessing expression of Fzd8, expression of Wnt2 expression may also be desired. Co-existence of Fzd8 expression elevated above that of a non-cancerous cell and expression of Wnt2 is a particular indicator that the cell is cancerous or at least pre-cancerous. As with Fzd8, the nucleic acid sequence of Wnt2 is known, and primers and probes to facilitate expression-based analysis of Wnt2 can be readily designed. Wnt2 mRNA can be assayed using methods known in the art, including those exemplified above for Fzd8. Wnt2 expression can be assayed by the same method or a different method as Fzd8 expression. For example, the mRNA levels of Fzd8 in a cell can be assayed for by RT-PCR, and the mRNA levels of Wnt2 in a cell can be assayed for by Northern blot, dot blot, microarray, in situ or RT-PCR. It may be desirable to use the same method for assaying Fzd8 and Wnt2 expression.

Detecting Fzd8/Wnt2 Polypeptide in a Cell

The detection methods above for Fzd8 and Wnt2 expression can also be accomplished by detection of Fzd8 and Wnt2 polypeptides. For example, Fzd8 expression can be assessed by detecting a level of Fzd8 polypeptide in a test cell and comparing this level to that of a normal cell. Fzd8 polypeptide and Wnt2 polypeptide can be detected by methods such as Western blotting and immunohistochemistry. Using the polypeptide sequences available in the art and disclosed herein, one of skill in the art can generate antibodies that will specifically bind to the Fzd8 receptor polypeptide, particularly to an extracellularly accessible portion of a Fzd8 receptor polypeptide. In addition, antibodies to Fzd8 are commercially available (Santa Cruz Biotechnology, catalogue number sc-33504, sc-33505 and sc-74022). When using an antibody to detect Fzd8 or Wnt2, the cells or tissue can be live, fixed with a fixative, or frozen. The level of the Fzd8 polypeptide can be determined by comparing the level of Fzd8 polypeptide in a sample suspected of containing cancerous cells to the level of a Fzd8 polypeptide in sample taken from normal cells (e.g., obtained from the same patient.)

Detecting Fzd8 Activity in a Cell

Fzd8 expression can also be assessed by assaying for Fzd8 activity in a cell. The present disclosure provides for detecting the level of Fzd8 activity in a cancer cell. The level of Fzd8 activity can be detected by detecting a level of a downstream production of Fzd8 receptor activation. For example, in the Wnt signaling pathway, in the absence of a Frizzled signal, the Wnt pathway member β-catenin is phosphorlyated and degraded. However, in the presence of a Frizzled signal, β-catenin is not degraded and it is translocated to the nucleus where it facilitates transcription of its target gene. Thus, Wnt signaling activity results in an increase in β-catenin levels. The level of β-catenin can be determined by detecting the level of β-catenin polypeptide. The polypeptide or the mRNA can be detected by using any of the techniques described above for detecting the level of a polypeptide or mRNA. The level of β-catenin in a cancerous cell can be compared to the level of β-catenin in a normal cell, wherein higher β-catenin levels are indicative of an active Fzd8 signal. Preferably, the cancerous cells and the normal cells are taken from the same patient. This assay of Fzd8 activity can be a useful assay in the clinic in determining if a patient's cells are cancerous or predisposed to cancer.

Detecting Fzd8 in a Cancer Cell with dnWnt2

dnWnt2 peptides can also be used to assess a level of Fzd8 expression of a cell. Such methods can use a detectably labeled Wnt2 polypeptide. For example, dnWnt polypeptides can be detectably labeled by a radioactive label or may be provided as a dnWnt2 fusion polypeptide having a detectable marker (e.g, a fluorescent protein such as GFP, or an epitope tag). Examples of detectable labels include enzymes, radioisotopes, fluorescent compounds, chemiluminescent compounds, phosphorescent compounds, bioluminescent compounds and haptens (e.g. biotin).

The method is generally accomplished by contacting a labeled dnWnt2 with a sample suspected of containing cancerous cells under conditions suitable for binding of dnWnt2 to Fzd8 receptor, if present. The amount of binding can be detected and compared with a control sample, e.g., a sample known to contain non-cancerous tissues or cells of the same origin. An increased amount of dnWnt2 binding to the sample suspected of containing cancerous cells compared to a level of dnWnt2 binding to non-cancerous cells of the same origin is indicative of a Fzd8-expressing cancer or a predisposition to a Fzd8-expressing cancer.

The dnWnt2-based detection method can find use in detection of any Fzd8-expressing cancer cell. The cancer cell can be of lung cancer, colon cancer, breast cancer, liver cancer, kidney cancer, prostate cancer and skin cancer (e.g. melanoma) origin. For example, Fzd8-expressing lung cancers that can be detected by the dnWnt2-based detection method include, but are not necessarily limited to, non-small cell lung carcinoma (NSCLC), small cell lung cancer (SCLC), adenocarcinoma, squamous cell carcinoma and large cell carcinoma. Optionally, the method involves detection of Wnt2 polypeptide in the cell, where the presence of above-normal levels of Fzd8 and the presence of Wnt2 indicates the cells is a cancerous or pre-cancerous cell.

Such methods also find use in monitoring therapy, including monitoring patients after treatment with a cancer therapeutic or surgery. After treatment of cancer, whether by a therapeutic agent or by surgery, cells can be taken from the site of tumor remission and assayed for the presence of Fzd8 and Wnt2 in the sample. The levels of Wnt2 and/or Fzd8 in the sample can be used to determine if the cancer cells are present and pose risk of relapse. This information can also be used in determining further treatments for the eradication of the cancer, for example, administration of a dnWnt2 to the patient.

### Methods of Screening for Candidate Agents

The present disclosure also provides methods of screening for candidate agents for activity as inhibitors of Wnt2/Fzd8-mediated cell proliferation. Such methods can be accomplished in cell-based and cell-free assays.

For example, where the assay is a cell-based assay, the methods generally involve contacting a cell expressing Fzd8 with a candidate agent in the presence of Wnt2 or other Fzd8 receptor activating agent, and determining whether the candidate agent reduces the level of Fzd8 signaling in the cell when compared to a control cell (e.g., compared to levels of Fzd8 signaling in a cell contacted with a dnWnt2 and/or compared to Fzd8 signaling in a cell in the absence of the candidate agent). Such methods are generally carried out in the presence of a wild-type, mature Wnt2 polypeptide.

Detection of Fzd8 signaling can be accomplished by any convenient method. For example, Fzd8 activity can be assessed by examining the effect of activity of a secondary messenger of Fzd8 activity (e.g., as may be assessed by expression), such as β-catenin, as set out in the Examples section below. Expression can be assessed by detection of activity of a reporter gene operably linked to a promoter responsive to a Fzd8 signal. Exemplary reporter genes include luciferase, β-galactosidase, and fluorescent proteins (e.g., GFP, YFP, BFP, RFP, and the like). The reporter gene can be operably linked to the TCF/LEF promoter (Behrens et al., Nature (1996) 382(6592): 638-42) which is responsive to the Fzd8 signal. Candidate agents that facilitate a reduction in detection of a detectable signal provided by expression of the reporter gene are those that inhibit Fzd8-mediated signaling.

The present disclosure also provides for cell-free methods of screening for a candidate agent. For example, such cell-free assays may use, for example, isolated Fzd8 receptor, Fzd8-presenting membrane fragments obtained from Fzd8-expressing cells, or a synthetic lipid bilayer presenting a Fzd8 receptor. Such cell-free methods generally involve contacting Fzd8 with a candidate agent in the presence of a dnWnt2 peptide, and determining whether the candidate agent can compete for binding to Fzd8 in the presence of dnWnt2. Candidate agents that act as competitors for Fzd8 can be optionally further tested in a cell-based assay, and may be further tested for inhibiting binding of a wild-type Wnt2 in the presence or absence of a dnWnt2 in a cell-free or cell-based assay.

The present disclosure provides for a method of screening by surface plasmon resonance (SPR) (Morton et al., Meth. Enzym. (1998)295: 268). SPR is a method for determining binding affinity, and monitors biomolecular interactions in real time. For example, Fzd8 can be attached to the SPR matrix, and a candidate agent which binds to the Fzd8 can be assayed for its association/disassociation rate constants, equilibrium dissociation constants and affinity constants. These measurements can be compared with the measurements obtained by binding dnWnt2 to Fzd8. The candidate agents obtained by cell-free methods can be further screened in a cell based assay for the blocking or reduction of Fzd8 signaling.

The present disclosure also provides for methods of automated screening or high throughput screening. High throughput screening assays that mimic the activity of a dnWnt2 can be designed to find candidate agents from chemical libraries, for example, see U.S. Pat. No. 5,541,061. Many of the high throughput screening assays can also be automated. Robotic systems that that handle sample processing, pipetting, washing and detection of the results are appropriate for screening candidate agents that mimic the activity of a dnWnt2. An example of such an automated system is the Biomek 3000® from Beckman Coulter Instruments (Fullerton, Calif.).

### Kits

Kits of the present disclosure include those suitable for use in the therapeutic methods and/or the diagnostic methods of the present disclosure.

For example, kits for use in the therapeutic methods of the present disclosure include unit doses of the dnWnt2 peptides disclosed herein, usually in injectable or oral formulations. In such kits, in addition to the containers containing the unit doses will be an informational package insert describing the use and attendant benefits of the dnWnt2 in alleviating cancer or conditions associated with cancer. Compounds and unit doses are those described above. The kit can comprise a dnWnt2 formulation in a sterile vial or in a syringe, which formulation can be suitable for injection in a mammal, particularly a human. The present disclosure provides for a kit useful in carrying out the detection methods for cancer, or a predisposition to cancer. The type of cancer detected is lung, colon, breast, liver, kidney, prostate and melanoma. A kit used for detecting cancer or the predisposition to comprises a probe or probes, for detecting the presence or level of Fzd8 and/or Wnt2. The probe can be a nucleotide probe, an antibody probe or in the case of detecting Fzd8, a labeled dnWnt2 polypeptide can be used as a probe. The kit can also contain a control for the normal level of Wnt2 and/or Fzd8. The kit will also contain instructions for use.

### EXAMPLES

### Materials and Methods

The following materials and methods were used in the Examples below.

Cell Lines and Tissues

NSCLC cell lines NCI-A549 and NCI-H460 were obtained from American Type Culture Collections (ATCC) (Manassas, Va.) and cultured in RPMI 1640 medium. Human kidney epithelial cell line 293 and human kidney transfected epithelial cell line (293T) were obtained from ATCC and cultured in Dulbecco's modified Eagle's medium (DMEM). DU 145 (ATCC® HTB-81™) and A427 (ATCC No. HTB 53) cell lines were obtained from ATCC, OE19 cell line was obtained from the European Collection of Cell Culture (Salisbury, UK). All cell cultures were supplemented with 10% fetal bovine serum, penicillin (100 IU/ml), and streptomycin (100 μg/ml). Cells were cultured at 37° C. in a humid incubator with 5% CO₂.

Fresh lung tumor tissues and adjacent normal lung tissues from patients who underwent surgical resection for lung cancers were collected and snap-frozen in liquid nitrogen within an hour of surgery. Tissue samples were kept at −170° C. in a liquid nitrogen freezer before use. The study was approved by the Committee of Human Research at the University of California and informed consent was obtained from all patients.

Plasmid DNA Constructs

The dominant-negative Wnt2 construct was generated by PCR amplification of the full-length human Wnt2 cDNA using primers flanking the N-terminal domain from residues 1-278. The nucleotide sequence of dnWnt2 is provided by SEQ ID NO. 1. The polypeptide sequence of human dnWnt2 is provided by SEQ ID NO 2. The amplified cDNA fragment was then inserted into the pEGFP-N1 vector (BD Biosciences Clontech, Palo Alto, Calif.) upstream of the GFP epitope to generate the dnWnt2 construct.

TOPflash Assay

Cell lines 293, 293T and A549 were plated in 96-well plates with fresh media without antibiotics 24 hr before transfection. Lipofectamine 2000 (Invitrogen, Carlsbad, Calif.) was used to mediate co-transfection of pTOPflash (0.2 μg) or pFOPflash (0.2 μg) vectors with each of the Fzd plasmids co-transfected with or without the following expression constructs: Wnt2, dnhWnt2 and empty vectors pcDNA3.1 (Invitrogen, Carlsbad, Calif.) or pEGFP-N1 (each at 0.2 μg; 0.6 μg DNA in total), as indicated. The Renilla luciferase reporter vector, pRL-TK (0.02 μg) (Promega, Madison, Wis.) was simultaneously transfected as the control for transfection efficiency. TCF-mediated transcriptional activity was determined by the ratio of pTOPflash/pFOPflash luciferase activity, each normalized to the luciferase activities of the pRL-TK reporter. Cells were harvested 48 hr after transfection. Luciferase assays were carried out using the Dual-Luciferase Reporter Assay System (Promega, Madison, Wis.). The experiments were done in triplicate and repeated independently at least four times.

Western Blot Analysis

Whole cell lysates of cell lines were obtained with CytoBuster Protein Extraction Reagent (Novagen, Madison, Wis.). The proteins were separated on 4-15% gradient SDS-polyacrylamide gels and transferred to Immobilon-P membranes (Millipore, Bellerica, Mass.). The proteins were first bound with the following primary antibodies: β-catenin (Transduction Laboratories, Lexington, Ky., USA) and β-actin (Sigma Chemical, St. Louis, Mo.). Antigen-antibody complexes were detected by using an ECL blotting analysis system (GE Healthcare Bio-Sciences, Piscataway, N.J.).

RNA Extraction and Reverse Transcription-PCR

Total RNA from lung cancer cell lines, fresh lung cancer, and paired adjacent normal tissue was isolated using an RNA extraction kit (RNeasy; Qiagen, Valencia, Calif.). Reverse transcription-PCR was performed in a GeneAmp PCR system 9700 (Applied Biosystems, Foster City, Calif.) using SuperScript One-step RT-PCR with Platinum Taq (Invitrogen, Carlsbad, Calif.) according to the manufacturer's protocol. Primers for reverse transcription-PCR were obtained from Operon Technologies (Alameda, Calif.). Primer sequences for the human Wnt2 cDNA are 5′-GGATGCCAGAGCCCTGATGAATCTT-3′(Forward) (SEQ ID NO:18) and 5′-GCCAGCCAGCATGTCCTGAGAGTA-3′ (reverse) (SEQ ID NO:19). Primers for the FZD8 cDNA are 5′ GGACTACAACCGCACCGACCT-3′ (forward) (SEQ ID NO:20), and 5′ ACCACAGGCCGATCCAGAAGAC-3′(reverse) (SEQ ID NO:21). The housekeeping gene glyceraldehyde-3-phosphate dehydrogenase (GAPDH) was amplified as an internal control.

Quantitative Real-Time Reverse Transcription-PCR

Total RNA from indicated cell lines was isolated using Qiagen's RNeasy extraction method (Valencia, Calif.). First-strand cDNA was synthesized from total RNA by iScript cDNA synthesis (Bio-Rad, Hercules, Calif.) according to the manufacturer's instructions. Taqman RT-PCR analysis was performed on cDNA in a 384-well plate using Prism 7900HT real-time PCR System (Applied Biosystems, Foster City, Calif.). Hybridization probes and primers for Wnt2 and Fzd8 (inventoried, chosen from the online catalog) were purchased from Applied Biosystems (Foster City, Calif.). The expression of each gene was assayed in triplicate and normalized to GAPDH (Applied Biosystems, Foster City, Calif.).

Cell Proliferation Assay (MTS Assay)

Cell proliferation was determined using the CellTiter 96 AQueous One Solution Cell Proliferation Assay (Promega, Madison, Wis.). Briefly, A549 and H460 cells were plated in a 6-well plate 24 hr before transfection. Transient transfection was carried out using 4 μg of the dnWnt2 construct or the pEGFP—N1 empty vector. Twenty-four hours after transfection, cells were seeded in a 96-well plate at 500 cells per well and cultured for another 24 hr period before adding the CellTiter 96 Aqueous One solution (day 0). The assay was repeated daily for 4 consecutive days. Cell viability was measured at absorbance 490 nm. Each experiment was done in sixtuplicate and repeated at least three times.

Apoptosis Analysis

Cells were harvested by trypsinization and stained using Annexin V FITC Apoptosis Detection method (Oncogene Science, Cambridge, Mass.), according to the manufacturer's specifications. Stained cells were immediately analyzed by flow cytometry (FACScan; Becton Dickinson, Franklin Lake, N.J.). Early apoptotic cells with exposed phosphatidylserine bound to Annexin V-FITC but excluded propidium iodide (PI). Cells in necrotic or late apoptotic stages were labeled with both Annexin V-FITC and PI.

### Example 1

### Wnt2 Binds Frz8

In order to determine the specific Fzd receptor for Wnt2 all ten known Fzd receptors were assayed for their ability to induce T-Cell Factor (TCF)-dependent transcription in the presence of Wnt2. The TCF promoter was operably linked to the luciferase gene, so that when the Wnt pathway was activated by Wnt2 specifically binding and activating a specific Fzd receptor, this would elicit luciferase gene transcription which could be detected by luciferase activity in the TOPflash assay.

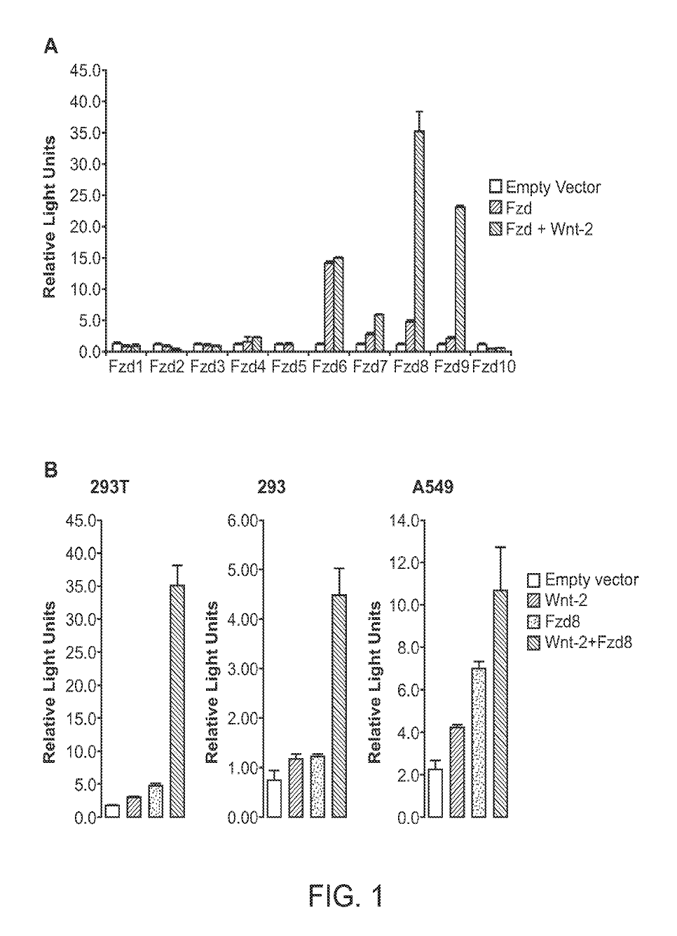

When Wnt2 was coexpressed with each of the ten Fzd receptors in 293T cells, a 25-fold increase in luciferase activity was observed for that of Fzd8 compared to vector alone (FIG. 1, panel A). Additionally a 16-fold increase in luciferase activity of Fzd9 over vector control alone was observed, similar to that previously reported (Gespach et al., FASEB J. (2005) 19(1):144-6). Fzd6 showed a 10-fold increase in luciferase activity with Wnt2, however there was no difference in TCF activity when transfected with Fzd6 vector alone and hence this activation cannot be due to the presence of Wnt2 alone. Fzd7 showed a 4-fold increase in TCF-activity compared to vector control and about a 2-fold increase due to the presence of Wnt2. None of the other Fzd expression vectors demonstrated significant activation due to Wnt2 coexpression (FIG. 1, panel A).

TOPflash TCF signaling was measured in 293 T cells transfected with the indicated Frizzled expression vectors in the presence or absence of Wnt2 cDNA and vector control (FIG. 1, panel A). Cells were cotransfected with pTOPflash or pFOPflash and internal control plasmid pRL-TK. Experiments were performed in triplicate and values represent the means (standard deviations) of three independent determinations expressed in relative luciferase units (RLU). Activation of Frizzled 8 by Wnt2 was measured in 293T, 293 and NSCLC cell line A549 (FIG. 1, panel B). TCF activity was determined in the cells transfected with Fzd8, Wnt2 or both Fzd8 and Wnt2 and vector control expression plasmids as described above. Experiments were performed in triplicate and the values represent the means (standard deviations) of at least three independent determinations expressed in RLU.

This activation was confirmed in Human Embryonic Kidney 293 cells and in the human lung cancer cell line A549. Wnt2 activation of Fzd 8 increased 5-fold in these cell lines compared to that of vector control (FIG. 1, panel B).

Immunoprecipitation analysis using recombinant Wnt-2-GST and Fzd8-Fc proteins was performed. Western blot analysis demonstrated binding between Wnt-2-GST and Fzd8-Fc but not with control IgG.

These results demonstrate the first reported interaction between Wnt2 and Fzd8.

### Example 2

### Inhibition of Fzd8 Activation by a Dominant Negative Wnt2

Because the Wnt pathway has been shown to have a role in cell proliferation and oncogenesis, a therapeutic that inhibited the Wnt pathway generated by the Wnt2/Fzd8 interaction would be useful in the reduction of cell proliferation. Thus, a Wnt2 that would bind Fzd8 but not induce a signal was designed.

Previous work in Xenopus showed that a C-terminal truncated Xwnt8 would bind its cognate Fzd receptor, but not induce a Wnt pathway signal, even in the presence of wild type Xwnt8. (Hoppler et al., Genes & Dev. (1996) 10:2805-2817). Such modified polypeptides that bind receptors and block signaling in the presence of wild type ligand are known in the art as dominant negative polypeptides.

Here, a Wnt2 containing a deletion of the carboxy-terminal region was designed. The human Wnt2 gene was truncated at amino acid position 278, deleting 82 carboxyl terminal amino acids. The dominant-negative Wnt2 construct was generated by PCR amplification of the full-length human Wnt2 cDNA using primers flanking the N-terminal domain from residues 1-278.

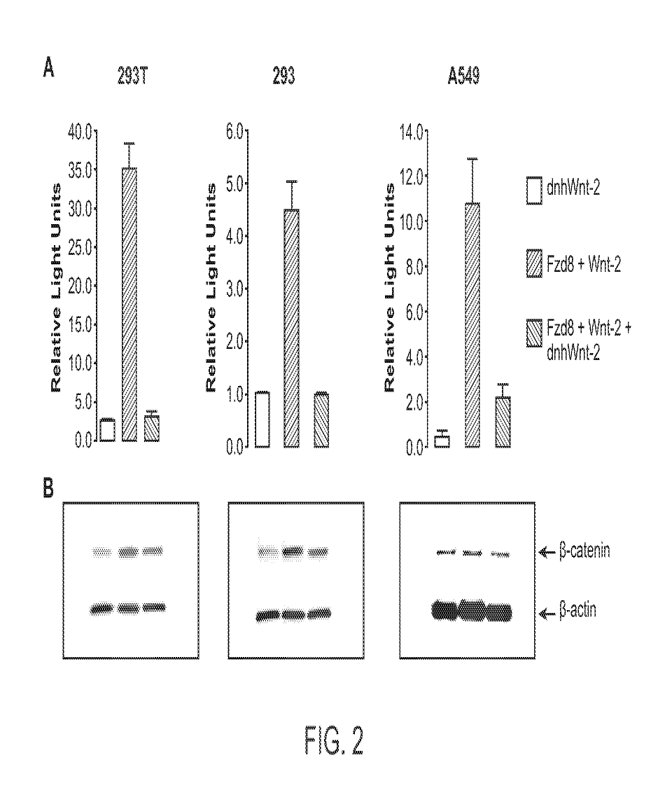

The dominant negative human Wnt2 (dnhWnt2) was co-transfected with wild type Wnt2 and Fzd8 in 293T, 293 and A549 cells. The amount of Wnt pathway activation was assayed using the TOPflash assay. In the cells expressing all three polypeptides there was a strong reduction in luciferase reporter activity compared to cells transfected wild type Wnt2/Fzd8. (FIG. 2, panel A).

dnhWnt2 inhibition of TCF-reporter transcriptional activity was measured in 293T, 293 and A549 cells that were cotransfected with either pTOPflash or pFOPflash and internal control plasmid pRL-TK, together with Fzd8 and Wnt2 expression vectors, in the presence or absence of the dnhWnt2 mutant (FIG. 2, panel A). Experiments were performed in triplicate and values represent the average of three independent determinations expressed in RLU. Wnt pathway inhibition by expression of the dnhWnt2 mutant was determined by Western blot analysis (FIG. 2, panel B) of β-catenin expression was performed on the cells treated as previously described.

Transfection of 293T cells with Fzd8 and Wnt2 produced a 25-fold level of activation which was reduced to near vector control levels by dnhWnt2. Similar inhibition was also observed with the other cell lines tested (FIG. 2, panel A). To confirm the dnhWnt2 was specific to the Wnt pathway, cytosolic β-catenin protein levels were analyzed. In cell lines coexpressing Fzd8 and wild type Wnt2 polypeptides, β-catenin protein levels were elevated, indicating activation of the Wnt signaling pathway. However coexpression of the dnhWnt2 polypeptide with Fzd8 and wild type Wnt2 polypeptides decreased β-catenin protein levels to that of control (FIG. 2, panel B.).

### Example 3

### Inhibition of Lung Cancer Cell Growth by dnWnt2

Because the dnhWnt2 potently inhibited Fzd8 signaling by Wnt2, experiments were performed to determine the endogenous levels of Wnt2 and Fzd8 in cell lines. These cell lines could then be used to determine if expression of dnhWnt2 could inhibit cell proliferation or induce apoptosis.

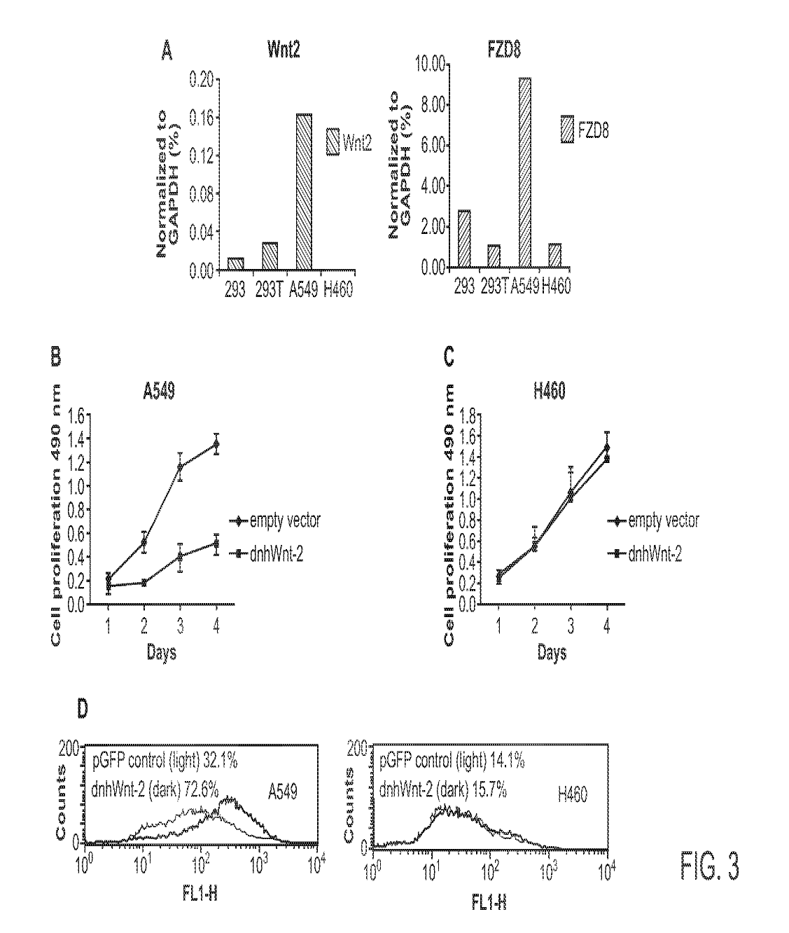

Endogenous expression levels of Wnt2 and Fzd8 were determined by quantitative real-time RT-PCR analysis in lung cancer cell lines A549 and H460 and cell lines 293 and 293T. Percent expression was normalized to GAPDH (FIG. 3. panel A).

The results were consistent with the expression patterns observed for Fzd8. Frizzled 8 was highly expressed in A549 cells, a finding also confirmed by previous reports (Saitoh et al., Int. J. Oncol. (2001) 18(5): 991-996). Wnt2 was highly expressed in NSCLC line A549. However, neither Fzd8 nor Wnt2 were expressed to a large extent in H460 cells. Expression levels of Wnt2 and Fzd8 were moderate in 293 and 293T cells (FIG. 3, panel A). Thus, the expression levels of both Wnt2 and Fzd8 were high in the NSCLC cell line A549, which was used to confirm the effectiveness of dnWnt2 as an inhibitor.

Cell proliferation was assessed in the cell line A549 (FIG. 3, panel B) and in the H460 cell line (FIG. 3, panel C) transfected with dnhWnt2 expression vector or empty vector. Cell viability was evaluated by MTS assay on days 1-4. Experiments were performed six times. Results are mean±Standard Deviation ((SD) error bars). The transfection of A549 cells with dnhWnt2 dramatically inhibited cell growth in these cells, as measured over a consecutive 4-day period (day 0 not shown) (FIG. 3, panel B). The lung cancer cell line H460, which does not highly express Wnt2 or Fzd8, was unaffected by transfections with dnhWnt2 (FIG. 3, panel C). These results demonstrated that dnhWnt2 could effectively inhibit Fzd8 signaling, and accordingly, inhibit cell proliferation, even in the presence of high levels of Wnt2. This demonstrates the effectiveness of dnhWnt2 as a Fzd8 inhibitor.

In order to determine if the inhibition of cell proliferation initiated apoptosis, the cells were subject to analysis by flow cytometry. A549 cells and H460 cells were transfected with dnhWnt2 expression vector or vector control and assayed with Annexin V and propidium iodide (FIG. 3, panel D; (Annexin V (FL1-H; X-axis) and PI (FL3-H; Y-axis) staining)) Experiments were performed in triplicate and a total of 20,000 cells were analyzed in each individual experiment.

This analysis revealed that dnhWnt2 significantly induced cell death in A549 cells (p=0.003) compared to A549 cells transfected with empty vector alone (FIG. 3, panel D (left)). In contrast, the H460 cells which do not highly express Wnt2 or Fzd8, the transfection with dnhWnt2 showed little apoptotic effect when compared to cells transfected with empty vector (FIG. 3, panel D (right)). The results demonstrated here show that the dnhWnt2 interacts with and suppresses signal transduction of Fzd8 by Wnt2, and that this inhibition leads to cancer cell death.

### Example 4

### Effect of dnhWnt2 on Colony Formation and TCF-Dependent Transcription in Cancer Cell Lines

Endogenous levels of Wnt-2 and Fzd8 in cancer cell lines, A427, DU145, OE19, were determined and compared to the expression levels of endogenous Wnt-2 and Fzd8 in A549 cancer cell line.

Real time PCR analysis confirmed high levels of both Wnt-2 and Fzd8 in lung cancer cell line A549. Real time PCR analysis showed that lung cancer cell line A427 express moderate levels of Wnt-2 and Fzd8. Prostate cancer cell line DU145 expressed Wnt-2 as determined by RT-PCR where as Barrett's esophageal cancer cell line OE19 did not and was used as a control.

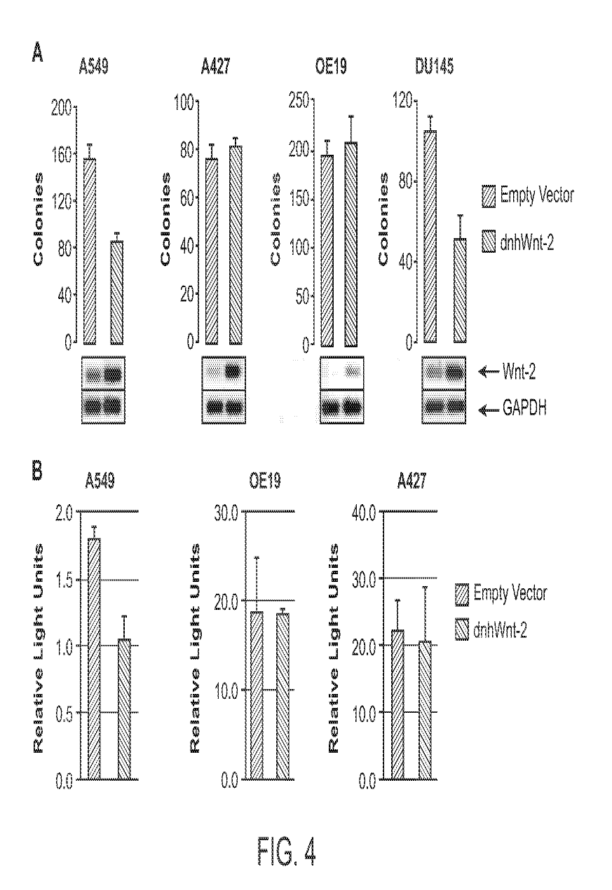

Stable cell lines containing the dnhWnt-2 construct were generated for the cancer cell lines. Stable cell lines containing empty control vector were generated for the cancer cell lines as controls. The numbers of colonies formed by the cell lines were determined visually. A549 and DU145 cells showed an about 52% and about 50% reduction in colony formation, respectively, upon dnhWnt-2 expression. Stable cell lines A427 and OE19 were unaffected by expression of the dnhWnt-2 gene (FIG. 4, panel A (Top)). RT-PCR analysis with primers specific to Wnt-2 confirmed expression of the dnhWnt-2 mutant (FIG. 4, panel A (bottom)).

TCF-mediated transcription assays were performed on the stable cell lines. A549 cells expressing the dnhWnt-2 gene showed a marked decrease in TCF-mediated transcription compared to vector control cells. Both A427 and OE19 cells were not affected by expression of the dnhWnt-2. (FIG. 4, panel B).

### Example 5

### A549 Xenograft Mouse Model

A xenograft mouse models were generated with A549 cells stably expressing the dnhWnt-2 gene or vector control.

A549 cells stably transfected with a vector expressing the dnhWnt-2 gene (A549 dnhWnt-2 stable cell line) or control vector (A549 empty vector stable cell line) were xenotransplanted into BALB/c-nude mice (n=5). Approximately one million cells were transplanted into the thigh and the shoulder of the mice.

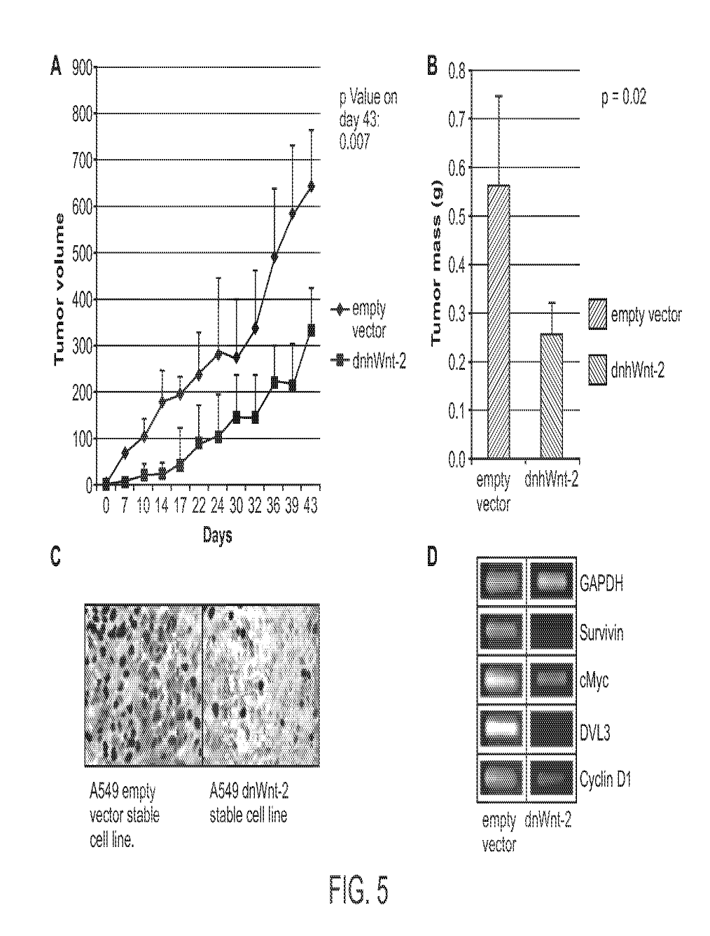

Tumor formation in the xenograft mouse models was monitored twice per week. The tumors formed by the A549 dnhWnt-2 stable cell line were significantly smaller in volume compared to tumors formed by the A549 empty vector stable cell line (FIG. 5, panel A; tumors formed by the A549 dnhWnt-2 stable cell line (squares) and tumors formed by the A549 empty vector stable cell line (circles). The tumors formed by the A549 dnhWnt-2 stable cell line were significantly smaller in mass compared to tumors formed by the A549 empty vector stable cell line (FIG. 5, panel B).

Tumors were removed from the xenograft mouse models at 43 days post-transplantation. Immunohistochemistry staining on tumor sections with Ki67 demonstrated a marked reduction in cell proliferation in tumor sections obtained from xenograft mouse models transplanted with A549 dnhWnt-2 stable cell line compared to cell proliferation in tumor sections obtained from xenograft mouse models transplanted with A549 empty vector stable cell line. Tumor sections obtained from xenograft mouse models transplanted with A549 empty vector stable cell line showed cell proliferation at approximately 80% compared to approximately 28% cell proliferation observed in tumor sections obtained from xenograft mouse models transplanted with A549 dnhWnt-2 stable cell line (>2000 cell counts) (FIG. 4, panel C).

RT-PCR analysis on Wnt-2 downstream target genes: survivin, c-Myc, Dvl-3 and Cyclin-D1 was performed on tumor sections removed from the xenograft mouse models at 43 days post-transplantation. RT-PCR analysis demonstrated that the expression of all of these genes were down-regulated in tumors formed by A549 dnhWnt-2 stable cell line compared to tumors formed by A549 empty vector stable cell line (FIG. 4, panel D).

### Example 6

### Expression of Wnt2 and Frz8 in Lung Cancer

The previous results demonstrated that dnhWnt2 was an effective inhibitor of Fzd8 signaling, causing a reduction in cell proliferation and eliciting apoptosis in cells that expressed Fzd8. Now knowing that cell proliferation could be reduced by dnhWnt2 in cells expressing Fzd8, 50 freshly resected lung cancer tumor samples were analyzed together with their corresponding matched normal lung pairs for Wnt2 and Fzd8 expression.

RNA from each tumor sample was reversed transcribed and amplified with the primers specific for Wnt2 or Fzd8. The data shown are representative tumor pairs (tumor (T) and normal (N)). PCR products were resolved on a 1.5% agarose gel. Experiments were performed at least twice.

The lung cancer tissues analyzed comprised 36 pairs of adenocarcinomas, 10 pairs of squamous cell carcinomas and 4 pairs of large cell carcinomas, and semiquanitative reverse transcription-polymerase chain reaction (RT-PCR) was performed on the tumor samples with their corresponding autologous, matched, normal lung controls.

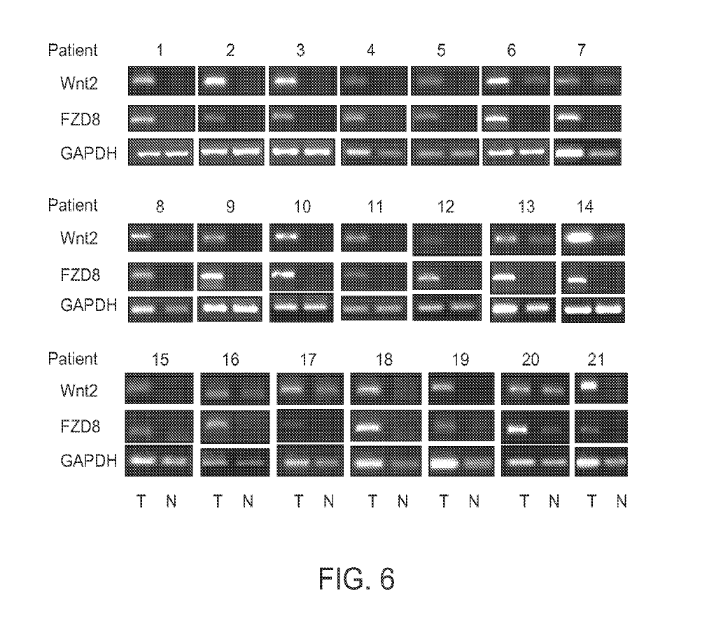

Wnt2 expression was up-regulated by 70% and human Fzd8 expression was up-regulated by 42% in the tumor samples when compared with the expression levels in their matched control samples. Furthermore, when the expression patterns of Wnt2 were compared with those for Fzd8, 91% of the lung cancer tumor samples showing increased expression of Fzd8 also showed concomitant up-regulation of Wnt2. (FIG. 6). This indicates that an increase in Fzd8 expression is often found in lung cancer. In addition, a large percentage of the lung cancers highly expressing Fzd8 also highly express Wnt2.

As was shown previously, dnhWnt2 is an effective inhibitor of lung cancer cell lines that express Wnt2 and Fzd8. This provides a reason to believe that dnhWnt2 would also inhibit cell proliferation in a primary lung cancer, given the result that a large percentage of lung cancers have a similar Fzd8 expression profile. Thus, the dnhWnt2 and the methods of the invention will prove useful as cancer therapeutics.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions.

That which is claimed is:

1. A dominant negative (dnWnt2) Wnt2 polypeptide comprising an amino acid sequence of a truncated Wnt2 polypeptide comprising five Wnt protein signature motifs, wherein the dnWnt2 polypeptide does not induce Fzd8 signal transduction and is capable of inhibiting induction of Fzd8 signal transduction mediated by a Wnt2 polypeptide.

2. The dnWnt2 of claim 1, which comprises a signal peptide at an N-terminus of the dnWnt2.

3. The dnWnt2 polypeptide of claim 1, wherein the dnWnt2 polypeptide is fused to a heterologous polypeptide.

4. The dnWnt2 polypeptide of claim 3, wherein the heterologous polypeptide comprises an epitope tag.

5. A nucleic acid comprising a nucleic acid sequence encoding a dnWnt2 polypeptide of claim 1.

6. A vector comprising the nucleic acid of claim 5.

7. A host cell comprising the nucleic acid of claim 5.

8. A method of inhibiting proliferation of a cancer cell expressing a Frizzled 8 (fzd8) receptor, the method comprising;
contacting a cancer cell expressing a Fzd8 receptor with the dnWnt2 polypeptide of claim 1 in an amount effective to inhibit Wnt2 polypeptide-mediated Fzd8 signal transduction;
wherein said contacting is effective to inhibit Wnt2-mediated cancer cell proliferation.

9. The method of claim 8, wherein the cancer cell is of lung, colon, breast, liver, kidney, prostate or skin origin.

10. The method of claim 8, wherein the cancer cell is present in a mammal having a Fzd8-expressing cancer.

11. A method of detecting a cancerous cell, the method comprising;
detecting a level of Frizzled 8 receptor (Fzd8) expression in a test cell,
wherein a level of Fzd8 in the test cell that is significantly elevated as compared to a level of Fzd8 expression of a normal cell indicates the test cell is cancerous.

12. The method of claim 11, comprising detecting a level of Wnt2 polypeptide in the cell, wherein a level of Wnt2 in the test cell that is significantly elevated as compared to a level of Wnt2 of a normal cell indicates the test cell is cancerous.

13. The method of claim 11, wherein the test cell is a lung, colon, breast, liver, kidney, prostate or skin cell.

14. The method of claim 11, wherein the test cell is a human cell.

15. A method of screening for a candidate agent for activity in inhibiting Frizzled 8 (Fzd8) receptor activation in the presence of a Wnt2 polypeptide, the method comprising:
contacting a cell that expresses Fzd8 with a candidate agent in the presence of a Wnt2 polypeptide;
detecting Fzd8 signaling in the cell;
wherein reduction of Fzd8 signaling in the presence of the candidate agent as compared to Fzd8 signaling in the absence of the candidate agent indicates the candidate agent has activity in inhibiting Wnt2-mediated Fzd8 signal transduction.

16. The method of claim 15, wherein the reduction of Fz8 signaling is determined by detecting a level of β-catenin polypeptide in the cell.

17. A method of screening for a candidate agent, the method comprising:
contacting a Fzd8 receptor with a candidate agent in the presence of a dnWnt2 polypeptide;
assaying the ability of the candidate agent to inhibit binding of the dnWnt2 polypeptide to the Fzd8 receptor;
wherein reduction of binding of dnWnt2 polypeptide in the presence of the candidate agent indicates that the candidate agent competes with dnWnt2 polypeptide for binding to the Fzd8 receptor.

18. The method of claim 17, wherein the dnWnt2 polypeptide is detectably labeled, and said assaying is by detecting a decrease in bound, detectably labeled dnWnt polypeptide in the presence of the candidate agent.

19. The method of claim 17, wherein the dnWnt2 polypeptide is detectably labeled, and said assaying is by detecting an increase in unbound detectably labeled Wnt2 polypeptide.

20. The method of claim 17, wherein said contacting is in the presence of a Wnt2 polypeptide.

21. The method of claim 20, wherein said Fzd8 receptor is expressed in a cell.
